(19) United States
(12) Patent Application Publication
(10) Pub. No.: US 2004/0208877 A1
(43) Pub. Date: Oct. 21, 2004

(54) Antibodies and uses thereof

(76) Inventors: Avigdor Levanon, Rehovot (IL); Rachel Ben-Levy, Reem (IL); Daniel Plaksin, Rehovot (IL); Esther Szanton, Rehovot (IL); Yocheved Hagai, Rehovot (IL); Hagit Hoch Mar-Chaim, Aseret (IL)

Correspondence Address: KENYON & KENYON, ONE BROADWAY, NEW YORK, NY, 10004, US

(21) Appl. No.: 10/611,588

(22) Filed: Jun. 30, 2003

Related U.S. Application Data
(60) Provisional application No. 60/393,491, filed on Jul. 1, 2002.

(51) Int. Cl. C12Q 1/68; A61K 39/395; C07K 16/40

(52) U.S. Cl. 424/146.1; 530/388.26

(57) ABSTRACT

The present invention provides antibodies or fragments thereof that bind to cancer cells and are important in physiological phenomena, such as cell rolling and metastasis. Therapeutic and diagnostic, prognostic or staging methods and compositions using such antibodies or fragments thereof are also provided. The methods and compositions according to the present invention can be used in diagnosis of and therapy for such diseases as cancer, including tumor growth and metastasis, leukemia, auto-immune disease, and inflammatory disease.

### FIELD OF THE INVENTION

The present invention relates to antibodies that bind to particular epitopes that are present on cells, such as cancer cells, metastatic cells, leukemia cells, leukocytes, and platelets, and that are important in such diverse physiological phenomena as cell rolling, metastasis, inflammation, and auto-immune diseases. Particularly, the antibodies may have anti-cancer activity, anti-metastatic activity, anti-leukemia activity, anti-viral activity, anti-infection activity, and/or activity against other diseases, such as inflammatory diseases, autoimmune diseases, cardiovascular diseases such as myocardial infarction, retinopathic diseases, and diseases caused by sulfated tyrosine-dependent protein-protein interactions.

### BACKGROUND OF THE INVENTION

Antibodies, Phase Display, and Tissue Targeting

Tissue-selective targeting of therapeutic agents is an emerging discipline in the pharmaceutical industry. New cancer treatments based on targeting have been designed to increase the specificity and potency of the treatment while reducing toxicity, thereby enhancing overall efficacy. Mouse monoclonal antibodies (MAbs) to tumor-associated antigens have been employed in an attempt to target toxin, radionucleotide, and chemotherapeutic conjugates to tumors. In addition, differentiation antigens, such as CD19, CD20, CD22, and CD25, have been exploited as cancer specific targets in treating hematopoietic malignancies.

Although extensively studied, this approach has several limitations. One limitation is the difficulty of isolating appropriate MAbs that display selective binding. A second limitation is the need for high antibody immunogenicity as a prerequisite for successful antibody isolation. A third limitation is that the final product has non-human sequences, which induce immune responses; e.g., when a mouse MAb is given to a human, a human anti-mouse antibody (HAMA) response will be generated. The HAMA response often results in a shorter serum half-life and prevents repetitive treatments, thus diminishing the therapeutic value of the antibody. This latter limitation has stimulated interest both in engineering chimeric or humanized monoclonal antibodies of murine origin and in discovering human antibodies. Another limitation of this approach is that it enables the isolation of only a single antibody species directed against only known and purified antigens. Moreover, this method is not selective insofar as it allows for the isolation of antibodies against cell surface markers that are present on normal, as well as malignant, cells.

There are many factors that influence the therapeutic efficacy of MAbs for treating cancer. These factors include the specificity and level of antigen expression on tumor cells, antigenic heterogeneity, and accessibility of the tumor mass. Leukemias and lymphomas have been generally more responsive to treatment with antibodies than solid tumors, such as carcinomas. MAbs rapidly bind to leukemia and lymphoma cells in the bloodstream and easily penetrate to malignant cells in lymphatic tissue, thus making lymphoid tumors excellent candidates for MAb-based therapy. An ideal system entails identifying a MAb that recognizes a marker on the cell surface of stem cells that are producing malignant progeny cells.

Phage libraries have been used to select random single chain variant fragments (scFvs) that bind to isolated, pre-determined target proteins, such as antibodies, hormones, and receptors. The use of antibody display libraries in general, and phage scFv libraries in particular, facilitates an alternative means of discovering unique molecules for targeting specific, yet unrecognized and undetermined, cell surface moieties.

Leukemia, lymphoma, and myeloma are cancers that originate in the bone marrow and lymphatic tissues and are involved in uncontrolled growth of cells. Acute lymphoblastic leukemia (ALL) is a heterogeneous disease that is defined by specific clinical and immunological characteristics. Like other forms of ALL, the definitive cause of most cases of B cell ALL (B-ALL) is not known; although, in many cases, the disease results from acquired genetic alterations in the DNA of a single cell, causing abnormalities and continuous multiplication. Prognosis for patients afflicted with B-ALL is significantly worse than for patients with other leukemias, both in children and in adults. Chronic lymphocytic leukemia (CLL), one example of which is B cell CLL (B-CLL), is a slowly progressing form of leukemia, characterized by an increased number of lymphocytes. Acute myelogenous leukemia (AML) is a heterogeneous group of neoplasms having a progenitor cell that, under normal conditions, gives rise to terminally differentiated cells of the myeloid series (erythrocytes, granulocytes, monocytes, and platelets). As in other forms of neoplasia, AML is associated with acquired genetic alterations that result in replacement of normally differentiated myeloid cells with relatively undifferentiated blasts, exhibiting one or more type of early myeloid differentiation. AML generally evolves in the bone marrow and, to a lesser degree, in the secondary hematopoietic organs. Primarily, AML affects adults and peaks in incidence between the ages of 15-40, but it is also known to affect both children and older adults. Nearly all patients with AML require treatment immediately after diagnosis to achieve clinical remission, in which there is no evidence of abnormal levels of circulating undifferentiated blast cells.

To date, a variety of MAbs have been developed that induce cytolytic activity against tumor cells. The murine MAb muMab4D5 produced against the extracellular domain of HER2 (P185) and found to markedly inhibit the proliferation of human tumor cells over-expressing HER2 was humanized to produce the drug HERCEPTIN® (trastuzumab), which was approved by the FDA and is being used to treat human breast cancer (U.S. Pat. Nos. 5,821,337 and 5,720,954). Following binding, the antibody is capable of inhibiting tumor cell growth that is dependent on the HER2 growth factor receptor. In addition, a chimeric antibody against CD20, which causes rapid depletion of peripheral B cells, including those associated with lymphoma, was recently approved by the FDA (U.S. Pat. No. 5,843,439). The binding of this antibody to target cells results in complement-dependent lysis. This product has recently been approved and is currently being used in the clinic to treat low-grade B cell non-Hodgkin's lymphoma.

Several other humanized and chimeric antibodies are under development or are in clinical trials. For example, a humanized immunoglobulin (Ig) that specifically reacts with CD33 antigen, expressed both on normal myeloid cells as well as on most types of myeloid leukemic cells, was conjugated to the anti-cancer drug calicheamicin, CMA-676 (Sievers et al., Blood Supp. 308: 504a (1997)). This conjugate, known as the drug MYLOTARG®, has recently received FDA approval (Caron et al., Cancer Supp. 73: 1049-56 (1994)). In light of its cytolytic activity, an additional anti-CD33 antibody (HumM195), currently in clinical trials, was conjugated to several cytotoxic agents, including the gelonin toxin (McGraw et al., Cancer Immunol. Immunother. 39: 367-74 (1994)) and radioisotopes ¹³¹I (Caron et al., Blood 83: 1760-68 (1994)), ⁹⁰Y (Jurcic et al., Blood Supp. 92: 613a (1998)) and ²¹³Bi (Humm et al., Blood Supplement 38: 231P (1997)). A chimeric antibody against the leukocyte antigen CD45 (cHuLym3) is also in clinical studies for treatment of human leukemia and lymphoma (Sun et al., Cancer Immunol. Immunother. 48: 595-602 (2000)). In in vitro assays, specific cell lysis was observed in ADCC (antibody dependent cell-mediated cytotoxicity) assays (Henkart, Immunity 1: 343-46 (1994); Squier and Cohen, Current Opin. Immunol. 6: 447-52 (1994)).

These therapeutic antibodies have also been specifically engineered to have higher affininity to their target, to be more stable, and for optimal biodistribution. See, e.g., Presta, Current Pharma. Biotechnol., 3: 237-56 (2002); Presta et al., Biochem. Society Transactions, 30(4): 487-90 (2002).

In contrast to mouse monoclonal humanization and construction of chimeric antibodies, the use of phage display technology enables the isolation of scFvs having fully human sequences. A fully human antibody against the human TGFβ2 receptor was recently developed based on a scFv clone derived from phage display technology. This scFv, which was converted into a fully human IgG4 capable of competing with the binding of TGFβ2 (Thompson et al., J. Immunol. Meth. 227: 17-29 (1999)), has strong anti-proliferative activity. Phage display technology, as known to one skilled in the art, is more specifically described in the following publications: Smith, Science 228: 1315 (1985); Scott et al., Science 249: 386-90 (1990); Cwirla et al., PNAS 87: 6378-82 (1990); Devlin et al., Science 249: 404-06 (1990); Griffiths et al., EMBO J. 13(14): 3245-60 (1994); Bass et al., Proteins 8: 309-14 (1990); McCafferty et al., Nature 348: 552-54 (1990); Nissim et al., EMBO J. 13: 692-98 (1994); U.S. Pat. Nos. 5,427,908, 5,432,018, 5,223,409 and 5,403,484, lib.

Lipand for Isolated scFv Antibody Molecules

Platelets, fibrinogen, GPIb, selectins, and PSGL-1 (P-Selectin Glycoprotein Ligand-1) each play an important role in several pathogenic conditions or disease states, such as abnormal or pathogenic inflammation, abnormal or pathogenic immune reactions, autoimmune reactions, metastasis, abnormal or pathogenic adhesion, thrombosis and/or restenosis, and abnormal or pathogenic aggregation. Thus, antibodies that bind to or cross-react with platelets and with these molecules would be useful in the diagnosis and treatment of diseases and disorders involving these and other pathogenic conditions.

Platelets

Platelets are well-characterized components of the blood system and play several important roles in hemostasis, thrombosis and/or restenosis. Damage to blood vessel sets in motion a process known as hemostasis, which is characterized by a series of sequential events. The initial reaction to damaged blood vessels is the adhesion of platelets to the affected region on the inner surface of the vessel. The next step is the aggregation of many layers of platelets onto the previously adhered platelets, forming a hemostatic plug and sealing the vessel wall. The hemostatic plug is further strengthened by the deposition of fibrin polymers. The clot or plug is degraded only when the damage has been repaired.

Circulating platelets are cytoplasmic particles released from the periphery of megakaryocytes. Platelets play an important role in hemostasis. Upon vascular injury, platelets adhere to damaged tissue surfaces and attach to one another (cohesion). This sequence of events occurs rapidly, forming a structureless mass (commonly called a platelet plug or thrombus) at the site of vascular injury. The cohesion phenomenon, also known as aggregation, may be initiated in vitro by a variety of substances, or agonists, such as collagen, adenosine-diphosphate (ADP), epinephrine, serotonin, and ristocetin. Aggregation is one of the numerous in vitro tests performed as a measure of platelet function.

Importance of Platelets in Metastasis

Tumor metastasis is perhaps the most important factor limiting the survival of cancer patients. Accumulated data indicate that the ability of tumor cells to interact with host platelets represents one of the indispensable determinants of metastasis (Oleksowicz, Thrombosis Res. 79: 261-74 (1995)). When metastatic cancer cells enter the blood stream, multicellular complexes composed of platelets and leukocytes coating the tumor cells are formed. These complexes, which may be referred to as microemboli, aid the tumor cells in evading the immune system. The coating of tumor cells by platelets requires expression of P-selectin by the platelets.

It has been demonstrated that the ability of tumor cells to aggregate platelets correlates with the tumor cells' metastasis potential, and inhibition of tumor-induced platelet aggregation has been shown to correlate with the suppression of metastasis in rodent models. It has been demonstrated that tumor cell interaction with platelets involves membrane adhesion molecules and agonist secretion. Expression of immuno-related platelet glycoproteins has been identified on tumor cell lines. It was demonstrated that platelet immuno-related glycoproteins, GPIb, GPIIb/IIIa, GPIb/IX and the integrin αᵥ subunit are expressed on the surface of breast tumor cell lines (Oleksowicz, (1995), supra; Kamiyama et al., J. Lab. Clin. Med. 117(3): 209-17 (1991)).

Gasic et al. (PNAS 61:46-52 (1968)) showed that antibody-induced thrombocytopenia markedly reduced the number and volume of metastases produced by CT26 colon adenocarcinoma, Lewis lung carcinoma, and B16 melanoma (Karpatkin et al., J. Clin. Invest. 81(4): 1012-19 (1988); Clezardin et al., Cancer Res. 53(19): 4695-700 (1993)). Furthermore, a single polypeptide chain (60 kd) was found to be expressed on surface membrane of HEL cells that is closely related to GPIb and corresponds to an incompletely or abnormally O-glycosylated GPIbα subunit (Kieffer et al., J. Biol. Chem. 261(34): 15854-62 (1986)).

GPIb Complex

Each step in the process of hemostasis requires the presence of receptors on the platelet surface. One receptor that is important in hemostasis is the glycoprotein Ib-IX complex (also known as CD42). This receptor mediates adhesion (initial attachment) of platelets to the blood vessel wall at sites of injury by binding von Willebrand factor (vWF) in the subendothelium. It also has crucial roles in two other platelet functions important in hemostasis: (a) aggregation of platelets induced by high shear in regions of arterial stenosis and (b) platelet activation induced by low concentrations of thrombin.

The GPIb-IX complex is one of the major components of the outer surface of the platelet plasma membrane. This complex comprises three membrane-spanning polypeptides—a disulfide-linked 130 kDa α-chain and 25 kDa β-chain of GPIb and a noncovalently associated GPIX (22 kDa). All of the subunits are presented in equimolar amounts on the platelet membrane for efficient cell-surface expression and function of CD42 complex, indicating that proper assembly of the three subunits into a complex is required for full expression on the plasma membrane. The α-chain of GPIb consists of three distinct structural domains: (1) a globular N-terminal peptide domain containing leucine-rich repeat sequences and Cys-bonded flanking sequences; (2) a highly glycosylated mucin-like macroglycopeptide domain; and (3) a membrane-associated C-terminal region that contains the disulfide bridge to GPIbα and transmembrane and cytoplasmic sequences.

Several lines of evidence indicate that the vWF and thrombin-binding domain of the GPIb-IX complex reside in a globular region encompasing approximately 300 amino acids at the amino terminus of GPIbα. As human platelet GPIb-IX complex is a key membrane receptor mediating both platelet function and reactivity, recognition of subendothelial-bound vWF by GPIb allows platelets to adhere to damaged blood vessels. Further, binding of vWF to GPIbα also induces platelet activation, which may involve the interaction of a cytoplasmic domain of the GPIb-IX with cytoskeleton or phospolipase A2. Moreover, GPIbα contains a high-affinity binding site for α-thrombin, which facilitates platelet activation by an as-yet poorly defined mechanism.

The N-terminal globular domain of GPIbα contains a cluster of negatively charged amino acids. Several lines of evidence indicate that in transfected CHO cells expressing GPIb-IX complex and in platelet GPIbα, the three tyrosine residues contained in this domain (Tyr-276, Tyr-278, and Tyr-279) undergo sulfation.

Protein Sulfation

Protein sulfation is a widespread post-translational modification that involves enzymatic covalent attachment of sulfate, either to sugar side chains or to the polypeptide backbone. This modification occurs in the trans-Golgi compartment. Sulfated proteins include secretory proteins, proteins targeted for granules, and the extracellular regions of plasma membrane proteins. Tyrosine is an amino acid residue presently known to undergo sulfation. Kehoe et al., Chem. Biol. 7: R57-61 (2000). Other amino acids, e.g., threonine, may also undergo sulfation, particularly in diseased cells.

A number of proteins have been found to be tyrosine-sulfated, but the presence of three or more sulfated tyrosines in a single polypeptide, as was found on GPIb, is not common. GPIbα (CD42), which is expressed by platelets and megakaryocytes and mediates platelet attachment to and rolling on subendothelium via binding with vWF, also contains numerous negative charges at its N-terminal domain. Such a highly acidic and hydrophilic environment is thought to be a prerequisite for sulfation, because tyrosylprotein sulfotransferase specifically recognizes and sulfates tyrosines adjacent to acidic amino residues (Bundgaard et al., J. Biol. Chem. 272:21700-05 (1997)). Full sulfation of the acidic region of GPIbα yields a region with a remarkable negative charge density—13 negative charges within a 19 amino acid stretch—and is a candidate site for electrostatic interaction with other proteins.

It is also thought that sulfated N-terminal tyrosines influence the role of CC-chemokine receptors, such as CCR5, which serve as co-receptors with related seven transmembered segment (7TMS) receptor for entry of human and simian immunodeficiency viruses (HIV-1, HIV-2, and SIV) into target cells. For example, it is thought that sulfated N-terminal tyrosines contribute to the binding of CCR5 to MIP-1α, MIP-1β, and HIV-1 gp120/CD4 complexes and to the ability of HIV-1 to enter cells expressing CCR5 and CD4. CXCR4, another important HIV-1 co-receptor, is also sulfated (Farzan et al., Cell 96(5): 667-76 (1999)). Tyrosine sulfation plays a less significant role in CXCR4-dependent HIV-1 entry than CCR5-dependent entry; thus demonstrating a possible role for tyrosine sulfation in the CXC-chemokine family and underscores a general difference in HIV-1 utilization of CCR5 and CXCR4 (Farzan et al., J. Biol. Chem. 277(33): 29,484-89 (2002)).

Selectins and PSGL-1

The P-, E-, and L- Selectins are members of a family of adhesion molecules that, among other functions, mediate rolling of leukocytes on vascular endothelium. P-Selectin is stored as granules in platelets and is transported to the surface after activation by thrombin, histamine, phorbol ester, or other stimulatory molecules. P-Selectin is also expressed on activated endothelial cells. E-Selectin is expressed on endothelial cells, and L-Selectin is expressed on neutrophils, monocytes, T cells, and B cells.

PSGL-1 (also called CD162) is a mucin glycoprotein ligand for P-Selectin, E-Selectin, and L-Selectin that shares structural similarity with GPlb (Afshar-Kharghan et al. (2001), supra). PSGL-1 is a disulfide-linked homodimer that has a PACE (Paired Basic Amino Acid Converting Enzymes) cleavage site. PSGL-1 also has three potential tyrosine sulfation sites followed by 10-16 decamer repeats that are high in proline, serine, and threonine. The extracellular portion of PSGL-1 contains three N-linked glycosylation sites and has numerous sialylated, fucosylated 0-linked oligosaccharide branches (Moore et al., J. Biol. Chem. 118: 445-56 (1992)). Most of the N-glycan sites and many of the O-glycan sites are occupied. The structures of the O-glycans of PSGL-1 from human HL-60 cells have been determined. Subsets of these O-glycans are core-2, sialylated and fucosylated structures that are required for binding to selectins. Tyrosine sulfation of an amino-terminal region of PSGL- 1 is also required for binding to P-Selectin and L-Selectin. Further, there is an N-terminal propeptide that is probably cleaved post-translationally.

PSGL-1 has 361 residues in HL60 cells, with a 267 residue extracellular region, 25 residue trans-membrane region, and a 69 residue intracellular region, and forms a disulfide-bonded homodimer or heterodimer on the cell surface (Afshar-Kharghan et al., Blood 97: 3306-12 (2001)). The sequence encoding PSGL-1 is in a single exon, so alternative splicing should not be possible. However, PSGL-1 in HL60 cells, and in most cell lines, has 15 consecutive repeats of a 10 residue consensus sequences present in the extracellular region, although there are 14 and 16 repeats of this sequence in polymorphonuclear leukocytes, monocytes, and several other cell lines, including most native leukocytes.

PSGL-1 is expressed on neutrophils as a dimer, with apparent molecular weights of both 250 kDa and 160 kDa, whereas on HL60 the dimeric form is approximately 220 kDa. When analyzed under reducing conditions, each subunit is reduced by half. Differences in molecular mass may be due to polymorphisms in the molecule caused by the presence of different numbers of decamer repeats (Leukocyte Typing VI. Edited by T. Kishimoto et al. (1997)).

Most blood leukocytes, such as neutrophils, monocytes, leukocytes, subset of B cells, and all T cells express PSGL-1 (Kishimoto et al. (1997), supra). PSGL-1 mediates rolling of leukocytes on activated endothelium, on activated platelets, and on other leukocytes and inflammatory sites and mediates rolling of neutrophils on P-Selectin. PSGL-1 may also mediate neutrophil-neutrophil interactions via binding with L-Selectin, thereby mediating inflammation (Snapp et al., Blood 91(1): 154-64 (1998)).

Leukocyte rolling is important in inflammation, and interaction between P-Selectin (expressed by activated endothelium and on platelets, which may be immobilized at sites of injury) and PSGL-1 is instrumental for tethering and rolling of leukocytes on vessel walls (Ramachandran et al., PNAS 98(18): 10166-71 (2001); Afshar-Kharghan et al. (2001), supra). Cell rolling is also important in metastasis, and P- and E-Selectin on endothelial cells is believed to bind metastatic cells, thereby facilitating extravasation from the blood stream into the surrounding tissues.

Thus, PSGL-1 has been found on all leukocytes: neutrophils, monocytes, lymphocytes, activated peripheral T cells, granulocytes, eosinophils, platelets, and on some CD34 positive stem cells and certain subsets of B cells. P-Selectin is selectively expressed on activated platelets and endothelial cells. Interaction between P-Selectin and PSGL-1 promotes rolling of leukocytes on vessel walls, and abnormal accumulation of leukocytes at vascular sites results in various pathological inflammations. Stereo-specific contributions of individual tyrosine sulfates on PSGL-1 are important for the binding of P-Selectin to PSGL-1. Charge is also important for binding: reducing NaCl (from 150 to 50 mM) enhanced binding (Kd ˜75 nM). Tyrosine-sulfation on PSGL-1 enhances, but is not ultimately required for PSGL-1 adhesion on P-Selectin. PSGL-1 tyrosine sulfation supports slower rolling adhesion at all shear rates and supports rolling adhesion at much higher shear rates (Rodgers et al., Biophys. J. 81: 2001-09 (2001)). Moreover, it has been suggested that PSGL-1 expression on platelets is 25-100 fold lower than that of leukocytes (Frenette et al., J. Exp. Med. 191(8): 1413-22 (2000)).

A commercially available monoclonal antibody to human PSGL-1, KPL1, has been shown to inhibit the interactions between PSGL-1 and P-selectin and between PSGL-1 and L-selectin. The KPL1 epitope was mapped to the tyrosine sulfation region of PSGL-1 (YEYLDYD) (Snapp et al., Blood 91(1):154-64 (1998)).

Pretreatment of tumor cells with O-sialoglycoprotease, which removes sialylated, fucosylated mucin ligands, also inhibited tumor cell- platelet complex formation. In vivo experiments indicate that either of these treatments results in greater monocyte association with circulating tumor cells, suggesting that reducing platelet binding increases access by immune cells to circulating tumor cells (Varki and Varki, Braz. J. Biol. Res. 34(6): 711-17 (2001)).

Fibrinogen

There are two forms of normal human fibrinogen—normal (γ) and γ prime, each of which is found in normal individuals. Normal fibrinogen, which is the more abundant form (approximately 90% of the total fibrinogen found in the body), is composed of two identical 55 kDa α chains, two identical 95 kDa β chains, and two identical 49.5 kDa γ chains. Normal variant fibrinogen, which is the less abundant form (approximately 10% of the fibrinogen found in the body), is composed of two identical 55 kDa α chains, two identical 95 kDa β chains, one 49.5 kDa γ chain, and one variant 50.5 kDa γ prime chain. The gamma and gamma prime chains are both coded for by the same gene, with alternative splicing occurring at the 3′ end. Normal gamma chain is composed of amino acids 1-411 and normal variant gamma prime chain is composed of 427 amino acids, of which amino acids 1-407 are the same as those in the normal gamma chain and amino acids 408-427 are VRPEHPAETEYDSLYPEDDL. This region is normally occupied with thrombin molecules.

Fibrinogen is converted into fibrin by the action of thrombin in the presence of ionized calcium to produce coagulation of the blood. Fibrin is also a component of thrombi, and acute inflammatory exudates.

Objectives

It is an object of the present invention to provide antibodies, fragments thereof or complexes thereof, that bind to epitopes present on various molecules instrumental in processes such as cell rolling, inflammation, immune reactions, infection, autoimmune reactions, and metastasis, yet are not involved in processes such as adhesion, thrombosis and/or restenosis and aggregation, which epitopes are present on diseased cells, such as AML cells, T-ALL cells, Pre-B-ALL cells, B-leukemia, B-CLL cells, multiple myeloma cells, and metastatic cells.

Another objective of the present invention includes the use of such antibodies in the development and provision of medicaments for the inhibition of cell rolling, inflammation, immune reactions, infection, autoimmune reactions, and metastasis, yet are not involved in adhesion, thrombosis and/or restenosis and aggregation, and for the treatment of diseases, such as AML, T-ALL, B-leukemia, B-CLL, Pre-B-ALL, multiple myeloma, metastasis, cardiovascular diseases such as myocardial infarction, retinopathic diseases, diseases caused by sulfated tyrosine-dependent protein-protein interactions, or other diseases in which such cellular functions or actions play a significant role.

It is also an object of this invention to utilize the antibodies in methods for diagnosing, prognosing, or staging various disease states of an individual, such as, e.g., AML, T-ALL, B-leukemia, B-CLL, Pre-B-ALL, multiple myeloma, and metastasis or other diseases in which such cellular functions or actions as cell rolling, inflammation, immune reactions, infection, autoimmune reactions, metastasis, play a significant role. And another object of the present invention is to provide a method of purging tumor cells.

Yet another object of the invention is to provide methods of activating ADCC or stimulating NK or T cells by administering the antibodies.

These and other objectives of the invention are provided herein.

### SUMMARY OF THE INVENTION

The present invention provides antibodies or fragments thereof having the binding capabilities of an scFv antibody fragment of SEQ ID NO: 1. The present invention also provides antibodies or fragments thereof, wherein at least one antibody, or binding fragment thereof, has a first hypervariable region of SEQ ID NO:2, a second hypervariable region of SEQ ID NO:3, and/or a third hypervariable region of SEQ ID NO:4. The antibodies or fragments thereof, of the present invention preferably bind to, or crossreact with, an epitope of PSGL-1. Also, preferably, the antibodies or fragments thereof of the present invention bind to an epitope on at least one cell type selected from the group consisting of T-ALL, AML, B-leukemia, B-CLL and multiple myeloma leukemia cells.

The present invention also provides isolated epitopes having an amino acid sequence that binds to the antibodies or binding fragments thereof of the present invention. Preferably, the isolated epitope is located between amino acids 1 and 17 of the mature PSGL-1, which is within a cluster of negatively charged amino acids.

Also provided are pharmaceutical compositions and processes for production of such antibodies or fragments thereof. Methods utilizing such pharmaceutical compositions to treat various conditions are provided, including conditions related to inhibiting or treating cell rolling; inhibiting or treating inflammation; inhibiting or treating an auto-immune disease; inhibiting or treating an infection (e.g., a viral infection such as HIV); inhibiting or treating metastasis; inhibiting or treating growth and/or replication of tumor cells; increasing mortality of tumor cells; inhibiting growth and/or replication of leukemia cells; increasing the mortality rate of leukemia cells; alters the susceptibility of diseased cells to damage by anti-disease agents; increasing the susceptibility of tumor cells to damage by anti-cancer agents; increasing the susceptibility of leukemia cells to damage by anti-leukemia agents; inhibiting increase in number of tumor cells in a patient having a tumor; decreasing the number of tumor cells in a patient having cancer; inhibiting increase in number of leukemia cells in a patient having leukemia; and decreasing the number of leukemia cells in a patient having leukemia. Other methods are provided to induce ADCC or stimulate NK or T cells using the present antibodies or fragments thereof.

The present invention also provides a method of purging tumor cells from a patient by providing a sample containing cells from the patient and incubating the cells from the patient with an antibody or polypeptide of the present invention.

### DEFINITIONS

Antibodies (Abs), or immunoglobulins (Igs), are protein molecules that bind to antigen. Each functional binding unit of naturally occurring antibodies is composed of units of four polypeptide chains (2 heavy and 2 light) linked together by disulfide bonds. Each of the chains has a constant and variable region. Naturally occurring antibodies can be divided into several classes including IgG, IgM, IgA, IgD, and IgE, based on their heavy chain component. The IgG class encompasses several sub-classes including, but not restricted to, IgG₁, IgG₂, IgG₃, and IgG₄. Immunoglobulins are produced in vivo by B lymphocytes, and each such molecule recognizes a particular foreign antigenic determinant and facilitates clearing of that antigen.

Antibodies may be produced and used in many forms, including antibody complexes. As used herein, the term “antibody complex” or “antibody complexes” is used to mean a complex of one or more antibodies with another antibody or with an antibody fragment or fragments, or a complex of two or more antibody fragments. Examples of antibody fragments include Fv, Fab, F(ab′)₂, Fc, and Fd fragments. Accordingly, the term “antibody or fragment thereof” as used herein includes an antibody complex or antibody complexes.

As used herein in the specification and in the claims, an Fv is defined as a molecule that is made up of a variable region of a heavy chain of a human antibody and a variable region of a light chain of a human antibody, which may be the same or different, and in which the variable region of the heavy chain is connected, linked, fused, or covalently attached to, or associated with, the variable region of the light chain. The Fv can be a single chain Fv (scFv) or a disulfide stabilized Fv (dsFv). An scFv is comprised of the variable domains of each of the heavy and light chains of an antibody, linked by a flexible amino-acid polypeptide spacer, or linker. The linker may be branched or unbranched. Preferably, the linker is 0-15 amino acid residues, and most preferably the linker is (Gly₄Ser)₃.

The Fv molecule, itself, is comprised of a first chain and a second chain, each chain having a first, second and third hypervariable region. The hypervariable loops within the variable domains of the light and heavy chains are termed Complementary Determining Regions (CDRs). There are CDR1, CDR2, and CDR3 regions in each of the heavy and light chains. These regions are believed to form the antigen binding site and can be specifically modified to yield enhanced binding activity. The most variable of these regions in nature is the CDR3 region of the heavy chain. The CDR3 region is understood to be the most exposed region of the Ig molecule and, as shown and provided herein, is the site primarily responsible for the selective and/or specific binding characteristics observed.

A fragment of an Fv molecule is defined as any molecule smaller than the original Fv that still retains the selective and/or specific binding characteristics of the original Fv. Examples of such fragments include but are limited to (1) a minibody, which comprises a fragment of the heavy chain only of the Fv, (2) a microbody, which comprises a small fractional unit of antibody heavy chain variable region (International Application No. PCT/IL99/00581), (3) similar bodies having a fragment of the light chain, and (4) similar bodies having a functional unit of a light chain variable region. It should be appreciated that a fragment of an Fv molecule can be a substantially circular or looped polypeptide.

As used herein the term “Fab fragment” is a monovalent antigen-binding fragment of an immunoglobulin. A Fab fragment is composed of the light chain and part of the heavy chain.

An F(ab′)₂ fragment is a bivalent antigen binding fragment of an immunoglobulin obtained by pepsin digestion. It contains both light chains and part of both heavy chains.

An Fc fragment is a non-antigen-binding portion of an immunoglobulin. It contains the carboxy-terminal portion of heavy chains and the binding sites for the Fc receptor.

An Fd fragment is the variable region and first constant region of the heavy chain of an immunoglobulin.

Polyclonal antibodies are the product of an immune response and are formed by a number of different B lymphocytes. Monoclonal antibodies are derived from one clonal B cell.

A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed, or attached at one or both ends.

The term “epitope” is used herein to mean the antigenic determinant or recognition site or antigen site that interacts with an antibody, antibody fragment, antibody complex or a complex having a binding fragment thereof or T cell receptor. The term epitope is used interchangeably herein with the terms ligand, domain, and binding region.

Selectivity is herein defined as the ability of a targeting molecule to choose and bind one entity or cell state from a mixture of entities or entity states, all entities or entity states of which may be specific for the targeting molecule.

The term “affinity” as used herein is a measure of the binding strength (association constant) between a binding molecule (e.g., one binding site on an antibody) and a ligand (e.g., antigenic determinant). The strength of the sum total of noncovalent interactions between a single antigen-binding site on an antibody and a single epitope is the affinity of the antibody for that epitope. Low affinity antibodies bind antigen weakly and tend to dissociate readily, whereas high-affinity antibodies bind antigen more tightly and remain bound longer. The term “avidity” differs from affinity, because the former reflects the valence of the antigen-antibody interaction.

Specificity of antibody-antigen interaction: Although the antigen-antibody reaction is specific, in some cases antibodies elicited by one antigen can cross-react with another unrelated antigen. Such cross-reactions occur if two different antigens share a homologous or similar structure, epitope, or an anchor region thereof, or if antibodies specific for one epitope bind to an unrelated epitope possessing similar structure conformation or chemical properties.

A platelet is a disc-like cytoplasmic fragment of a megakaryocyte that is shed in the marrow sinus and subsequently circulates in the peripheral blood stream. Platelets have several physiological functions including a major role in clotting. A platelet contains centrally located granules and peripheral clear protoplasm, but has no definite nucleus.

Agglutination as used herein means the process by which suspended bacteria, cells, discs, or other particles of similar size are caused to adhere and form into clumps. The process is similar to precipitation but the particles are larger and are in suspension rather than being in solution.

The term aggregation means a clumping of platelets induced in vitro, and thrombin and collagen, as part of a sequential mechanism leading to the formation of a thrombus or hemostatic plug.

Conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical substitutions that may be performed for such conservative amino acid substitution may be among the groups of amino acids as follows:

glycine (G), alanine (A), valine (V), leucine (L) and isoleucine (I)

aspartic acid (D) and glutamic acid (E)

alanine (A), serine (S) and threonine (T)

histidine (H), lysine (K) and arginine (R)

asparagine (N) and glutamine (Q)

phenylalanine (F), tyrosine (Y) and tryptophan (W)

Conservative amino acid substitutions can be made in, e.g., regions flanking the hypervariable regions primarily responsible for the selective and/or specific binding characteristics of the molecule, as well as other parts of the molecule, e.g., variable heavy chain cassette. Additionally or alternatively, modification can be accomplished by reconstructing the molecules to form full-size antibodies, diabodies (dimers), triabodies (timers), and/or tetrabodies (tetramers) or to form minibodies or microbodies.

A phagemid is defined as a phage particle that carries plasmid DNA. Phagemids are plasmid vectors designed to contain an origin of replication from a filamentous phage, such as m13 of fd. Since it carries plasmid DNA, the phagemid particle does not have sufficient space to contain the full complement of the phage genome. The component that is missing from the phage genome is information essential for packaging the phage particle. In order to propagate the phage, therefore, it is necessary to culture the desired phage particles together with a helper phage strain that complements the missing packaging information.

A promoter is a region on DNA at which RNA polymerase binds and initiates transcription.

A phage display library (also termed phage peptide/antibody library, phage library, or peptide/antibody library) comprises a large population of phages (10⁸ or larger), each phage particle displaying a different peptide or polypeptide sequence. These peptide or polypeptide fragments may constructed to be of variable length. The displayed peptide or polypeptide can be derived from, but need not be limited to, human antibody heavy or light chains.

A pharmaceutical composition refers to a formulation which comprises an antibody or peptide or polypeptide of the invention and a pharmaceutically acceptable carrier, excipient or diluent thereof, or an antibody-pharmaceutical agent (antibody-agent) complex and a pharmaceutically acceptable carrier, excipient or diluent thereof.

A agent refers to an agent that is useful in the treatment of active disease, prophylactic treatment, or diagnosis of a mammal including, but not restricted to, a human, bovine, equine, porcine, murine, canine, feline, or any other warm-blooded animal. The agent is selected from the group of radioisotope, toxin, pharmaceutical agent, oligonucleotide, recombinant protein, antibody fragment, anti-cancer agents, anti-adhesion agents, anti-thrombosis agents, anti-restenosis agents, anti-autoimmune agents, anti-aggregation agents, anti-bacterial agents, anti-viral agents, and anti-inflammatory agents. Other examples of such agents include, but are not limited to anti-viral agents including acyclovir, ganciclovir, and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid.

An anti-leukemia agent is an agent with anti-leukemia activity. For example, anti-leukemia agents include agents that inhibit or halt the growth of leukemic or immature pre-leukemic cells, agents that kill leukemic or pre-leukemic cells, agents that increase the susceptibility of leukemic or pre-leukemic cells to other anti-leukemia agents, and agents that inhibit metastasis of leukemic cells. In the present invention, an anti-leukemia agent may also be an agent with anti-angiogenic activity that prevents, inhibits, retards or halts vascularization of tumors.

The expression pattern of a gene can be studied by analyzing the amount of gene product produced under various conditions, at specific times, in various tissues, etc. A gene is considered to be “over-expressed” when the amount of gene product is higher than that found in a normal control, e.g., non-diseased control.

A given cell may express on its surface a protein having a binding site (or epitope) for a given antibody, but that binding site may exist in a cryptic form (e.g., be sterically hindered or blocked, or lack features needed for binding by the antibody) in the cell in a state, which may be called a first stage (stage I). Stage I may be, e.g., a normal, healthy, non-diseased status. When the epitope exists in cryptic form, it is not recognized by the given antibody, i.e., there is no binding of the antibody to this epitope or to the given cell at stage I. However, the epitope may be exposed by, e.g., undergoing modifications itself, or being unblocked because nearby or associated molecules are modified or because a region undergoes a conformational change. Examples of modifications include changes in folding, changes in post-translational modifications, changes in phospholipidation, changes in sulfation, changes in glycosylation, and the like. Such modifications may occur when the cell enters a different state, which may be called a second stage (stage II). Examples of second states, or stages, include activation, proliferation, transformation, or in a malignant status. Upon being modified, the epitope may then be exposed, and the antibody may bind.

Peptido-mimetics (peptide mimetics) are molecules (e.g., antibodies) that no longer contain any peptide bonds, i.e., amide bonds, between amino acids; however, in the context of the present invention, the term peptide mimetic is intended to include molecules that are no longer completely peptidic in nature, such as pseudo-peptides, semi-peptides and peptoids. Whether completely or partially non-peptide, peptidomimetics according to this invention provide a spatial arrangement of reactive chemical moieties that closely resembles the three-dimensional arrangement of active groups in the peptide on which the peptidomimetic is based. These molecules include small molecules, lipids, polysaccharides, or conjugates thereof.

### BRIEF DESCRIPTION OF THE DRAWINGS

FIG. 1 depicts FACS analysis following TM3.13 scFv staining of T-ALL cells.

FIG. 2 depicts numerical data from Lumiaggregometer analysis of platelet aggregation in the presence of scFv antibodies as a percent of control aggregation of either washed platelets or PRP.

FIG. 3 depicts FACS analysis comparing binding of various scFv antibodies to platelets: FIG. 3A is AN51-PE (FSC), FIG. 3B is AN51-PE (FL2-H), FIG. 3C is a negative control, FIG. 3D is Y1-myc+, FIG. 3E is Y1, FIG. 3F is L32, and FIG. 3G is TM1.1.

FIG. 4 depicts FACS analyses that compare the ability of scFv antibodies (N01, Y1-myc+, and L32) to compete and interfere with the binding of labeled Y1 antibody to KG-1 cells at different concentrations: FIG. 4A is 0 ng, FIG. 4B is 100 ng, FIG. 4C is 250 ng, FIG. 4D is 500 ng, FIG. 4E is 1000 ng, FIG. 4F is 2500 ng, and FIG. 4G is 5000 ng.

FIG. 5 depicts numerical data from FACS analyses that compare the ability of scFv antibodies (N0 1, Y 1 -myc+, and L32) to compete and interfere with the binding of labeled Y1 antibody to KG-1 cells.

FIG. 6 depicts numerical data from ELISA analyses providing comparisons of the binding of different concentrations of scFv antibodies (TM1.1, Y1 -myc+, Y1, and L32) to glycocalicin.

FIG. 7 depicts Western analysis of L32 and Y1 scFv antibody binding to GC, plasma, and membranal proteins from KG-1 and Raji cells.

FIG. 8 depicts numerical data from ELISA analyses providing comparisons of the binding of scFv antibodies (Y1-myc+, TM1.1, and L32) to fibrinogen, PSGL-1, and GPIbα-related peptides.

FIG. 9 depicts numerical data from FACS analyses following staining of platelets with Y17 scFv antibody, in the presence of varying concentrations of GPIb-derived peptides. The results are presented as percent reduction in the geo mean of the response obtained with the scFv antibody alone.

FIG. 10 depicts numerical data from ELISA analyses following binding of Y1 and Y17 scFv antibody to PSGL1 sulfated at various different positions.

FIG. 11 depicts numerical data from FACS analyses providing comparisons of the binding of scFv antibodies (FIG. 11A is TM1.1, FIG. 11B is TM1.3, and FIG. 11C is L32) to T-ALL and normal peripheral blood cells (N-PBL) as a function of the concentration of each scFv antibody.

FIG. 12 depicts numerical data from an in vivo study of the effects of L32 and Y1 scFv antibody administration on livers weights (FIG. 12A) and tumor prevalence (FIG. 12B) in a SCID mice-Molt4 cells tumor model.

### DETAILED DESCRIPTION OF THE INVENTION

The present invention relates to an antibody or fragment thereof having binding capabilities of an scFv antibody fragment of SEQ ID NO: 1, that binds PSGL-1. Thus, these antibodies of the present invention have similar binding affinity as SEQ ID NO: 1. The scFv fragment of SEQ ID NO: 1 has been designated L32. Accordingly, preferably, an antibody of the present invention is L32. This antibody was identified by screening a phage library, which has diversity only in the heavy chain CDR3 regions, against a leukemia cell to select specific antibodies that recognize leukemia cell surface determinants, wherein the specific receptor was not previously known or characterized. Using this same method, another antibody, L3 1, was identified. Athough the present invention encompasses many antibodies, L32 will be used hereafter exemplarily.

Previously, other antibodies that bind to leukemic cells were identified in U.S. application Ser. Nos. 10/032,423; 10/032,037; 10/029,988; 10/029,926; 09/751,181; and 60/258,948 and International Application Nos. PCT/US01/49442 and PCT/US01/49440 using the same phage library. Specific examples of antibodies disclosed in these applications include the Y1 and Y17 antibodies. The antibodies disclosed in these applications were discovered to specifically bind to an epitope, found on proteins of the hematopoetic cells, which is sulfated at an N-terminal tyrosine and is thought to be involved in cell migration, e.g., tumor metastasis.

Both the L32 antibody and the antibodies disclosed in the Y1/Y17 applications bind leukemic cells, although L32 binds to leukemic cells with approximately five times greater affinity than Y1. Based on this fact, as well as the fact that the antibodies were isolated from a common germ line (DP32), comparison studies were done to determine the correlation between their respective binding epitopes. It was subsequently determined that L32 appears to bind the same sulfated epitope as Y1/Y17. As the Y1/Y17 epitope is specifically present on platelets, although it has been suggested that the levels of expression are 25-100 fold lower than that of leukocytes (Frenette et al., J. Exp. Med. 191(8): 1413-22 (2000)), binding of L32 to platelets was also evaluated. However, it was found that L32 only negligably binds platelets and, moreover, does not affect platelet aggregation. Set forth in Table 1 is a summary of Y1 scFv and IgG as compared to L32 scFv and IgG.

TABLE 1

| | Y1 scFv | Y1 IgG | L32 scFv | L32 IgG |
|---|---|---|---|---|
| WBC Binding | Low | High | High | Very High |
| Leukemia Cell | Low | High | High | Very High |
| Binding | | | | |
| PSGL-1 | Low | High | High | Very High |
| Reactivity | | | | |
| Competition | Somewhat | Yes | Yes | Yes |
| with KPL1 | | | | |
| Platelet | High | High | Low-None | Low-None |
| Binding | | | | |
| Platelet | Inhibits | Induction | No Effect | — |
| Aggregation | | | | |
| GPIb | Binds | Binds | Very Low- | Very Low- |
| Reactivity | | | None | None |
| Plasma | Fibrinogen γ | Fibrinogen γ | Not Detected | Very Low- |
| Component | prime | prime | | None |
| | CCF4 | CCF4 | | |
| In vitro Effect | Not Detected | ADCC | Not Detected | ADCC |

The sulfated epitopes previously identified as binding to Y1/Y17 are characterized by the presence of sulfated moieties, such as sulfated tyrosine residues or sulfated carbohydrate or lipid moieties, preferably within a cluster of two or more acidic amino acids, which are found on ligands and receptors that play important roles in such diverse processes as inflammation, immune reactions, infection, autoimmune reactions, metastasis, adhesion, thrombosis and/or restenosis, cell rolling, and aggregation. Such epitopes are also found on diseased cells, such as B-leukemia cells, B-CLL cells, AML cells, multiple myeloma cells, and metastatic cells. These epitopes are useful targets for the therapeutic mediation of these processes and for diagnostic or prognostic procedures, including staging.

The L32 scFv has enhanced selectivity for sulfated PSGL-1. White cells involved in inflammation, such as monocytes, neutrophils, and lymphocytes, are primarily recruited by the four adhesion molecules, PSGL-1, P-selectin, VLA-4, and VCAM-1 in the inflammatory processes of diseases such as atherosclerosis (Huo and Ley, Acta Physiol. Scand., 173: 35-43 (2001); Libby, Sci. Am. May: 48-55 (2002); Wang et al., J. Am. Coll. Cardiol. 38: 577-582 (2001)). L32 interference with any of these central molecules may suggest a potential role for L32 in abrogating related diseases. Specifically, P-selectin controls cell attachment and rolling. Additionally, P-selectin—PSGL-1 interactions activate a number of other molecules on cells which are integrally connected with tumorigenesis (when concerned with malignant cells) and inflammatory responses (when concerned with white blood cells) (Shebuski and Kilgore, J. Pharmacol. Exp. Ther. 300: 729-735 (2002)). Based on this understanding of P-selectin's ability to regulate cellular processes, it is apparent that the enhanced L32 scFv selectivity for sulfated PSGL-1 may make it a superior molecule for treating a variety of malignant and inflammatory diseases. Moreover, models of malignant disease have shown that P-selectin binding to malignant cells requires sulfation of PSGL-1 (Ma and Geng, J. Immunol. 168: 1690-1696 (2002)). This requirement is similar to that for L32 binding. Thus, one can expect that L32 could abrogate P-selectin facilitation of progressing malignant disease.

Moreover, it was found for these antibodies of the present invention that binding is dependent on the stage of development of the cell (AML subtype is classified based on the French-American-British system using the morphology observed under routine processing and cytochemical staining): the antibodies bind to AML cells that are of subtype M3 or above, but not M0 or M1 subtype cells. In addition, the antibodies may or may not bind M2 subtype cells. Accordingly, the antibodies of the present invention do not bind normal, healthy bone marrow (e.g., CD34+ cells). It is thought that such differences are based on alterations in PSGL-1 expression and/or sulfation, as well as possible conformational changes in PSGL-1 that expose a slightly different epitope.

Preferably, the L32 antibody of the present invention binds different molecules or epitopes involved in inflammation, such as PSGL-1. Also preferably, the L32 antibody binds to an epitope present on at least one cell type involved in inflammation or tumorogenesis, including T-ALL cells, AML cells, and B-leukemia cells. Further preferably, the L32 antibody of the present invention binds to epitopes on a lipid, carbohydrate, peptide, glycolipid, glycoprotein, lipoprotein, and/or lipopolysaccharide molecule. Such epitopes preferably have at least one sulfated moiety. Alternatively, but also preferably, the L32 antibody cross-reacts with two or more epitopes, each epitope having one or more sulfated tyrosine residues, and at least one cluster of two or more acidic amino acids, an example of which is PSGL-1.

Following binding to PSGL-1 present on the surface of a cell, some of these antibodies or fragment thereof of the present invention may be internalized into the cell. Generally, full IgG antibodies are internalized, while smaller antibody fragments (such as scFvs) are not internalized. It should be appreciated that the antibodies can be internalized into any cell expressing PSGL-1, inlcuding AML cells, for example. Such internalization may occur via endocytosis, an active process that is manner, time and temperature dependent.

It is the hypervariable regions of the L32 antibodies of the present invention that participate in forming the antigen binding sites. The antigen-binding site is complementary to the structure of the epitopes to which the antibodies bind, therefore these binding sites are referred to as complementarity-determining regions (CDRs). There are three CDRs on each light and heavy chain of an antibody (CDR1, CDR2, and CDR3), each located on the loops that connect the β strands of the V_{H} and V_{L} domains. The most variable of these regions is the CDR3 region of the heavy chain. The CDR3 region is understood to be the most exposed region of the Ig molecule and, as provided herein, has a central role in determining the selective and/or specific binding characteristics observed.

DP32, which is one of the 49 germ lines present in the phage display library, is the specific germ line of the phage library from which the scFv antibodies of the present invention were isolated. Therefore, DP32 provides the antibodies of the present invention with at least the heavy and light chain framework variable regions, light chain CDR1, CDR2, and CDR3 regions, and/or heavy chain CDR1 and CDR2. DP32 also provides a three-dimensional structure on which the hypervariable regions were conformed. It is well known that the specificity of an antibody is determined by its three-dimensional conformation. Thus, the limitations imposed by DP32 may have a significant role in determining the specificity of L32 antibodies. Moreover, DP32 has various charged amino acids, which may have a structural role in L32 antibody recognition.

According to the present invention, CDRs may be inserted into cassettes to produce antibodies. A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed, or attached at one or both ends.

The amino acid sequence of the cassette may ostensibly be fixed, whereas the replaced, inserted, or attached sequence can be highly variable. The cassette can be comprised of several domains, each of which encompasses a function crucial to the final construct.

The cassette of a particular embodiment of the present invention comprises, from the N-terminus, framework region 1 (FR1), CDR1, framework region 2 (FR2), CDR2, framework region 3 (FR3), and framework region 4 (FR4).

In an embodiment of the invention, it is possible to replace distinct regions within the cassette. For example, the CDR2 and CDR1 hypervariable regions of the cassette may be replaced or modified by non-conservative or, preferably, conservative amino acid substitutions.

In a preferred embodiment of the invention, the antibody or fragment thereof has a heavy and a light chain, and each chain has a first, second, and third hypervariable region, which are the CDR3, CDR2, and CDR1 regions, respectively. Particularly, a CDR3 region of one chain, either the CDR3 region of the light chain or, preferably, the CDR3 region of the heavy chain and, more preferably, both heavy and light chain CDR3 regions determine binding selectivity and specificity. Secondarily, the binding selectivity and specificity are determined by the CDR2 and CDR1 regions of the light chain and, preferably, of the heavy chain. The upstream or downstream regions flanking the first, second, and/or third hypervariable regions may also secondarily influence the binding selectivity and specificity.

In one preferred embodiment of the present invention, at least one antibody or fragment thereof has a first hypervariable region (CDR3) of SEQ ID NO:2. In addition, or alternatively, at least one antibody or fragment thereof has a second hypervariable region (CDR2) of SEQ ID NO:3. Also, in addition, or alternatively, at least one antibody pr fragment thereof has a third hypervariable region (CDR1) of SEQ ID NO:4. More preferably, at least one antibody or fragment thereof has a first hypervariable region (CDR3) of SEQ ID NO:2 and a second hypervariable region (CDR2) of SEQ ID NO:3 and a third hypervariable region (CDR1) of SEQ ID NO:4.

In a particularly preferred embodiment, at least one antibody, or binding fragment thereof, of the antibody or fragment thereof is an scFv having SEQ ID NO: 1.

For all of the amino acid sequences of <25 amino acid residues described and detailed herein (e.g., CDR regions, CDR flanking regions), it is to be understood and considered as a further embodiment of the invention that these amino acid sequences include within their scope one or two amino acid substitution(s) and that preferably the substitutions are conservative amino acid substitutions. For all of the amino acid sequences of >25 amino acid residues described and detailed herein, it is to be understood and considered as an embodiment of the invention that these amino acid sequences include within their scope an amino acid sequence with ≧90% sequence similarity to the original sequence (Altschul et al., Nucleic Acids Res. 25: 3389-402 (1997)). Similar or homologous amino acids are defined as non-identical amino acids which display similar properties, e.g., acidic, basic, aromatic, size, positively or negatively charged, polarity, non-polarity.

Percent amino acid similarity or homology or sequence similarity is determined by comparing the amino acid sequences of two different peptides or polypeptides. Antibody sequences were determined by DNA sequencing. The two sequences are aligned, usually by use of one of a variety of computer programs designed for the purpose, and amino acid residues at each position are compared. Amino acid identity or homology is then determined. An algorithm is then applied to determine the percentage amino acid similarity. It is generally preferable to compare amino acid sequences, due to the greatly increased sensitivity to detection of subtle relationships between the peptide, polypeptide or protein molecules. Protein comparison can take into account the presence of conservative amino acid substitutions, whereby a mismatch may yet yield a positive score if the non-identical amino acid has similar physical and/or chemical properties (Altschul et al. (1997), supra).

In an embodiment of the invention, the three hypervariable regions of each of the light and heavy chains can be interchanged between the two chains and among the three-hypervariable sites within and/or between chains. Moreover, the sequences of the hypervariable regions can be altered to span two or more of the CDRs. Also in framework variable regions—also so that may only partially be ine 1 CDR

The present invention provides for a peptide or polypeptide having an antibody or antigen binding fragment thereof, a construct thereof, or a construct of a fragment. According to the present invention, antibodies include IgG, IgA, IgD, IgE, or IgM antibodies. The IgG class encompasses several sub-classes including IgG₁, IgG₂, IgG₃, and IgG₄.

Antibodies may be provided in many forms, such as fragments, complexes, and multimers. According to the present invention, antibody fragments include Fv, scFv, dsFv, Fab, Fab₂, and Fd molecules. Smaller antibody fragments, such as fragments of Fvs and fragments of Fabs, are also included in the term “fragments”, as long as they retain the binding characteristics of the original antibody or larger fragment. Constructs include, for example, multimers such as diabodies, triabodies, and tetrabodies. The phrases “antibody, binding fragment thereof, or complex having an antibody or binding fragment thereof” and “antibody or fragment” are intended to encompass all of these molecules, as well as derivatives, combinations, modifications, homologs, mimetics, and variants thereof, unless it is specified otherwise or indicated otherwise based on context and/or knowledge in the art.

It has been established that scFv penetrate tissues and are cleared from the blood more rapidly than a full size antibody because they are smaller in size (Adams et al., Br. J. Cancer 77: 1405-12 (1988); Hudson, Curr. Opin. Immunol. 11(5): 548-557 (1999); Wu et al., Tumor Targeting 4: 47 (1999)). Thus, scFv are often employed in diagnostics involving radioactive labels such as tumor imaging to allow for a more rapid clearance of the radioactive label from the body. A number of cancer targeting scFv multimers have recently undergone pre-clinical evaluation for in vivo stability and efficacy (Adams et al. (1988), supra; Wu (1999), supra).

Typically, scFv monomers are designed with the C-terminal end of the V_{H} domain tethered by a polypeptide linker to the N-terminal residue of the V_{L}. Optionally an inverse orientation is employed: the C-terminal end of the V_{L} domain is tethered to the N-terminal residue of V_{H} through a polypeptide linker (Power et al., J. Immun. Meth. 242: 193-204 (2000)). The polypeptide linker is typically around fifteen amino acids in length. When the linker is reduced to about three to seven amino acids, the scFvs can not fold into a functional Fv domain and instead associate with a second scFv to form a diabody. Further reducing the length of the linker to less than three amino acids forces the scFv association into trimers or tetramers, depending on the linker length, composition and Fv domain orientations. (Powers (2000), supra).

Recently, it has been discovered that multivalent antibody fragments such as scFv dimers, trimers, and tetramers often provide higher affinity over the binding of the parent antibody to the target. This higher affinity offers potential advantages including improved pharmaco-kinetics for tumor targeting applications. Additionally, in studying P-Selectin and its ligand PSGL-1, which are involved in tethering and rolling of leukocytes, scientists have concluded that cells expressing dimeric forms of PSGL- 1 established more stable rolling adhesions because of this higher binding affinity. These adhesions are more sheer resistant and exhibited less fluctuation in rolling velocities. (Ramachandran et al., PNAS, 98(18): 10166-71 (2001)).

Mulitvalent forms of scFv have been designed and produced by others. One approach has been to link two scFvs with linkers. Another approach involves using disulfide bonds between two scFvs for the linkage. The simplest approach to production of dimeric or trimeric Fv was reported by Holliger et al., PNAS 90: 6444-48 (1993) and Kortt et al., Protein Eng. 10: 423-33 (1997). One such method was designed to make dimers of scFvs by adding a sequence of the FOS and JUN protein region to form a leucine zipper between them at the c-terminus of the scFv (Kostelny et al., J Immunol. 148(5): 1547-53 (1992); De Kruif et al., J Biol Chem. 271(13): 7630-34 (1996)). Another method was designed to make tetramers by adding a streptavidin coding sequence at the c-terminus of the scFv. Streptavidin is composed of 4 subunits, so when the scFv-streptavidin is folded, 4 subunits accommodate themselves to form a tetramer (Kipriyanov et al., Hum Antibodies Hybridomas 6(3): 93-101 (1995)). In yet another method, to make dimers, trimers, and tetramers, a free cysteine is introduced in the protein of interest. A peptide-based cross linker with variable numbers (2 to 4) of maleimide groups was used to cross link the protein of interest to the free cysteines (Cochran et al., Immunity 12(3): 241-50 (2000)).

The greater binding affinity of these multivalent forms may be beneficial in diagnostics, prognostics, staging, and therapeutic regimens. For example, a scFv may be employed as a blocking agent to bind a target receptor and thus block the binding of the “natural” ligand. In such instances, it is desirable to have a higher affinity association between the scFv and the receptor to decrease chances for disassociation, which may allow an undesirable binding of the natural ligand to the target. In addition, this higher affinity may be useful when the target receptors are involved in adhesion and rolling or when the target receptors are on cells present in areas of high sheer flow, such as platelets.

In this system, the phage library (as described herein above) can be designed to display scFvs, which can fold into the monovalent form of the Fv region of an antibody. Further, and also discussed herein above, the construct is suitable for bacterial expression. The genetically engineered scFvs comprise heavy chain and light chain variable regions joined by a contiguously encoded 15 amino acid flexible peptide spacer. The preferred spacer is (Gly₄Ser)₃. The length of this spacer, along with its amino acid, constituents provides for a nonbulky spacer, which allows the V_{H} and the V_{L} regions to fold into a functional Fv domain that provides effective binding to its target.

Varying the length of the spacers is yet another preferred method of forming dimers, trimers, and triamers (often referred to in the art as diabodies, triabodies, and tetrabodies, respectively). Dimers are formed under conditions where the spacer joining the two variable chains of a scFv is shortened to generally 5-12 amino acid residues. This shortened spacer prevents the two variable chains from the same molecule from folding into a functional Fv domain. Instead, the domains are forced to pair with complimentary domains of another molecule to create two binding domains. In a preferred method, a spacer of only 5 amino acids (Gly₄Ser) was used for diabody construction. This dimer can be formed from two identical scFvs, or from two different populations of scFvs and retain the selective and/or specific enhanced binding activity of the parent scFv(s), and/or show increased binding strength or affinity.

In a similar fashion, triabodies are formed under conditions where the spacer joining the two variable chains of a scFv is shortened to generally less than 5 amino acid residues, preventing the two variable chains from the same molecule from folding into a functional Fv domain. Instead, three separate scFv molecules associate to form a trimer. In a preferred method, triabodies were obtained by completely removing this flexible spacer. The triabody can be formed from three identical scFvs, or from two or three different populations of scFvs, and retain the selective and/or specific enhanced binding activity of the parent scFv(s), and/or show increased binding strength or affinity.

Tetrabodies are similarly formed under conditions where the spacer joining the two variable chains of a scFv is shortened to generally less than 5 amino acid residues, preventing the two variable chains from the same molecule from folding into a functional Fv domain. Instead, four separate scFv molecules associate to form a tetramer. The tetrabody can be formed from four identical scFvs, or from 1- 4 individual units from different populations of scFvs and should retain the selective and/or specific enhanced binding activity of the parent scFv(s), and/or show increased binding strength or affinity. Whether triabodies or tetrabodies form, under conditions where the spacer is generally less than 5 amino acid residues long, depends on the amino acid sequence of the particular scFv(s) in the mixture and the reaction conditions.

Once an antibody, fragment, or construct having desired binding capabilities has been selected and/or developed, it is well within the ability of one skilled in the art using the guidance provided herein to produce constructs and fragments which retain the characteristics of the original antibody. For example, full antibody molecules, Fv fragments, Fab fragments, Fab₂ fragments, dimers, trimers, and other constructs can be made which retain the desired characteristics of the originally selected or developed antibody, fragment, or construct.

If it is desired to substitute amino acids, but still retain the characteristics of an antibody or fragment, it is well within the skill in the art to make conservative amino acid substitutions. Modifications such as conjugating to various agents may also be made to antibodies or fragments without altering their binding characteristics. Other modifications, such as those made to produce more stable antibodies or fragments may also be made to antibodies or fragments without altering their specificity. For example, peptoid modification, semipeptoid modification, cyclic peptide modification, N terminus modification, C terminus modification, peptide bond modification, backbone modification, and residue modification may be performed. It is also within the ability of the skilled worker following the guidance of the present specification to test the modified antibodies or fragments to assess whether their binding characteristics have been changed.

Likewise, it is within the ability of the skilled worker using the guidance provided herein to alter the binding characteristics of an antibody, fragment, or construct to obtain a molecule with more desirable characteristics. For example, once an antibody having desirable properties is identified, random or directed mutagenesis may be used to generate variants of the antibody, and those variants may be screened for desirable characteristics.

Using conventional methods known in the art, one of skill would also be able to determine addition antibodies or fragments thereof that have the binding capabilities of the L32 scFv. For example, additional antibodies can be isolated using the biopanning methods described herein, wherein the molecule or cell that L32 binds is used to screen a particular phage display library, particularly a library prepared from a leukemia, lymphoma, and myeloma patient.

Antibodies and fragments, according to the present invention, may also have a tag that may be inserted or attached thereto to aid in the preparation and identification thereof, and in diagnostics or prognostics, including staging. The tag can later be removed from the molecule. Examples of useful tags include: AU1, AU5, BTag, c-myc, FLAG, Glu-Glu, HA, His6, HSV, HTTPHH, IRS, KT3, Protein C, S-TAG®, T7, V5, and VSV-G (Jarvik and Telmer, Ann. Rev. Gen., 32, 601-18 (1998)). The tag is preferably c-myc or KAK.

Antibodies, fragments thereof or constructs thereof, peptides, polypeptides, proteins, and fragments and constructs thereof can be produced in either prokaryotic or eukaryotic expression systems. Methods for producing antibodies and fragments in prokaryotic and eukaryotic systems are well-known in the art.

A eukaryotic cell system, as defined in the present invention and as discussed herein, refers to an expression system for producing peptides or polypeptides by genetic engineering methods, wherein the host cell is a eukaryote. A eukaryotic expression system may be a mammalian system, and the peptide or polypeptide produced in the mammalian expression system, after purification, is preferably substantially free of mammalian contaminants. Other examples of a useful eukaryotic expression system include yeast expression systems.

A preferred prokaryotic system for production of the peptide or polypeptide of the invention uses E. coli as the host for the expression vector. The peptide or polypeptide produced in the E. coli system, after purification, is substantially free of E. coli contaminating proteins. Use of a prokaryotic expression system may result in the addition of a methionine residue to the N-terminus of some or all of the sequences provided for in the present invention. Removal of the N-terminal methionine residue, after peptide or polypeptide production to allow for full expression of the peptide or polypeptide, can be performed as is known in the art, one example being with the use of Aeromonas aminopeptidase under suitable conditions (U.S. Pat. No. 5,763,215).

In a preferred embodiment of the present invention, the process for producing an antibody or fragment thereof has the steps of: (a) providing a phage display library; (b) providing a molecule or cell that an antibody or fragment thereof having the binding capabilities of an scFv antibody fragment of SEQ ID NO: 1 may bind to; (c) panning the phage display library for a phage particle displaying an oligopeptide or polypeptide that binds to the molecule or cell; and (d) producing an antibody or fragment thereof having at least one antibody or binding fragment thereof having an antibody or binding fragment thereof, having the peptide or polypeptide that binds to the molecule or cell.

The antibodies and polypeptides of the subject invention can be complexed with e.g. associated with, combined, fused, or linked to various pharmaceutical agents, such as drugs, toxins, and radioactive isotopes and optionally, with a pharmaceutically effective carrier, to form peptide-drug compositions comprising an antibody/polypeptide and a pharmaceutical agent having anti-disease and/or anti-cancer activity. Such compositions may also be used for diagnostic purposes

Examples of carriers useful in the invention include dextran, HPMA (a hydrophilic polymer), or any other polymer, such as a hydrophilic polymer, as well as derivatives, combinations and modifications thereof. Alternatively, decorated liposomes can be used, such as liposomes decorated with scFv Y1 molecules (e.g., Doxil, a commercially available liposome containing large amounts of doxorubicin). Such liposomes can be prepared to contain one or more desired agents and be admixed with the antibodies of the present invention to provide a high drug to antibody ratio.

Alternatively, the link between the antibody or fragment thereof and the agent may be a direct link. A direct link between two or more neighboring molecules may be produced via a chemical bond between elements or groups of elements in the molecules. The chemical bond can be, for example, an ionic bond, a covalent bond, a hydrophobic bond, a hydrophilic bond, an electrostatic bond, or a hydrogen bond. The bonds can be, for example, amide, carbon-sulfide, peptide, and/or disulfide bonds. In order to attach the the antibody to the agent or linker, amine, carboxy, hydroxyl, thiol and ester functional groups may be used, as is known in the art to form covalent bonds.

The link between the peptide and the agent or between the peptide and carrier, or between the carrier and agent may be via a linker compound. As used herein, in the specification and in the claims, a linker compound is defined as a compound that joins two or more moieties. The linker can be straight-chained or branched. A branched linker compound may be composed of a double-branch, triple branch, or quadruple or more branched compound. Linker compounds useful in the present invention include those selected from the group having dicarboxylic acids, malemido hydrazides, PDPH, carboxylic acid hydrazides, and small peptides.

More specific examples of linker compounds useful, according to the present invention, include: (a) dicarboxylic acids such as succinic acid, glutaric acid, and adipic acid; (b) maleimido hydrazides such as N-[maleimidocaproic acid] hydrazide, 4-[N-maleimidomethyl]cyclohexan-1-carboxylhydrazide, and N-[maleimidoundcanoic acid] hydrazide; (c) (3-[2-pyridyldithio]propionyl hydrazide); and (d) carboxylic acid hydrazides selected from 2-5 carbon atoms, and derivatives, combinations, modifications, and analogues thereof.

Linking via direct coupling using small peptide linkers is also useful. For example, direct coupling between the free sugar of, for example, the anti-cancer drug doxorubicin and a scFv may be accomplished using small peptides. Examples of small peptides include AU1, AU5, BTag, c-myc, FLAG, Glu-Glu, HA, His6, HSV, HTTPHH, IRS, KT3, Protein C, S-TAG®, T7, V5, VSV-G, and KAK.

Antibodies, and fragments thereof, of the present invention may be bound to, conjugated to, complexed with, or otherwise associated with imaging agents (also called indicative markers), such as radioisotopes, and these conjugates can be used for diagnostic, prognostic, or staging and imaging purposes. Kits having such radioisotope-antibody (or fragment) conjugates are provided.

Examples of radioisotopes useful for diagnostics, prognostics, or staging include ¹¹¹indium, ¹¹³indium, ^{99m}rhenium, 105rhenium, 101rhenium, ^{99m}technetium, ^{121m}tellurium, ^{122m}tellurium, ^{125m}telluriunm ¹⁶⁵thulium, ¹⁶⁷thulium ¹⁶⁸thulium ¹²³iodine, ¹²⁶iodine, 131iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵ruthenium, ¹⁰⁷mercury, ²⁰³mercury, ⁶⁷gallium, and ⁶⁸gallium. Preferred radioactive isotopes, are opaque to X-rays or any suitable paramagnetic ions.

The indicative marker molecule may also be a fluorescent marker molecule. Examples of fluorescent marker molecules include fluorescein, phycoerythrin, or rhodamine, or modifications or conjugates thereof.

Antibodies or fragments conjugated to indicative markers may be used to diagnose, prognose or stage disease states by providing a sample containing a cell from the patient and determining whether the antibodies of the present invention bind to the cell of the patient, thereby indicating that the patient is at risk for or has the disease. Moreover, the present invention also provides a method of purging tumor cells from a patient by providing a sample containing cells from the patient and incubating the cells from the patient with an antibody of the present invention. Such activities may be carried out in vivo, in vitro, or ex vivo. Where carried out in vivo or ex vivo, the imaging agent is preferably physiologically acceptable in that it does not harm the patient to an unacceptable level. Acceptable levels of harm may be determined by clinicians using such criteria as the severity of the disease and the availability of other options.

With respect to cancer, staging a disease in a patient generally involves determining the classification of the disease based on the size, type, location, and invasiveness of the tumor. One classification system to classify cancer by tumor characteristics is the “TNM Classification of Malignant Tumours” (6th Edition) (L. H. Sobin, Ed.), which is incorporated by reference herein and which classifies stages of cancer into T, N, and M categories with T describing the primary tumor according to its size and location, N describing the regional lymph nodes, and M describing distant metastases. In addition, the numbers I, II, III and IV are used to denote the stages and each number refers to a possible combination of TNM factors. For example, a Stage I breast cancer is defined by the TMN group: T1, N0, M0 which mean:T1—Tumor is 2 cm or less in diameter, N0—No regional lymph node metastasis, M0—No distant metastasis. Another system is used to stage AML, with subtypes of classified based on the French-American-British system using the morphology observed under routine processing and cytochemical staining.

In addition, a recently proposed World Health Organization (WHO) staging or classification of neoplastic diseases of the hematopoietic and lymphoid tissues includes (specifically for AMLs) traditional FAB-type categories of disease, as well as additional disease types that correlate with specific cytogenetic findings and AML associated with myelodysplasia. Others have also proposed pathologic classifications. For example, one proposal specific for AML includes disease types that correlate with specific cytogenetic translocations and can be recognized reliably by morphologic evaluation and immunophenotyping and that incorporate the importance of associated myelodysplastic changes. This system would be supported by cytogenetic or molecular genetic studies and could be expanded as new recognizable clinicopathologic entities are described (Arber, Am. J Clin. Pathol. 115(4): 552-60 (2001)).

The present invention provides for a diagnostic, prognostic, or staging kit for in vitro analysis of treatment efficacy before, during, or after treatment, having an imaging agent having a peptide of the invention linked to an indicative marker molecule, or imaging agent. The invention further provides for a method of using the imaging agent for diagnostic localization, prognostication of survival, and staging or imaging of a cancer, more specifically a tumor, having the following steps: (a) contacting the cells with the composition; (b) measuring the radioactivity bound to the cells; and hence (c) visualizing the tumor.

Examples of suitable imaging agents include fluorescent dyes, such as FITC, PE, and the like, and fluorescent proteins, such as green fluorescent proteins. Other examples include radioactive molecules and enzymes that react with a substrate to produce a recognizable change, such as a color change.

In one example, the imaging agent of the kit is a fluorescent dye, such as FITC, and the kit provides for analysis of treatment efficacy of cancers, more specifically blood-related cancers, e.g., leukemia, lymphoma, and myeloma. FACS analysis is used to determine the percentage of cells stained by the imaging agent and the intensity of staining at each stage of the disease, e.g., upon diagnosis, during treatment, during remission and during relapse.

Antibodies, and fragments thereof, of the present invention may be bound to, conjugated to, or otherwise associated with anti-cancer agents, anti-neoplastic agents, anti-viral agents, anti-metastatic agents, anti-inflammatory agents, anti-thrombosis agents, anti-restenosis agents, anti-aggregation agents, anti-autoimmune agents, anti-adhesion agents, anti-cardiovascular disease agents, pharmaceutical agents or other anti-disease agents. An agent refers to an agent that is useful in the prophylactic treatment or diagnosis of a mammal including, but not restricted to, a human, bovine, equine, porcine, murine, canine, feline, or any other warm-blooded animal.

Examples of such agents include, but are not limited to, anti-viral agents including acyclovir, ganciclovir and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid.

Exemplary pharmaceutical agents include the anthracyclines, such as doxorubicin (adriamycin), daunorubicin (daunomycin), idarubicin, detorubicin, carminomycin, epirubicin, esorubicin, as well as morpholino and substituted derivates and combinations thereof. Further exemplary pharmaceutical agents include cis-platinum, taxol, calicheamicin, vincristine, cytarabine (Ara-C), cyclophosphamide, prednisone, fludarabine, idarubicin, chlorambucil, interferon alpha, hydroxyurea, temozolomide, thalidomide and bleomycin, and derivatives, combinations and modifications thereof.

Inhibition of growth of a cancer cell includes, for example, (i) prevention of cancerous or metastatic growth, (ii) slowing down of the cancerous or metastatic growth, (iii) total prevention of the growth process of the cancer cell or the metastatic process, while leaving the cell intact and alive, (iv) interfering contact of cancer cells with the microenvironment, or (v) killing the cancer cell.

Inhibition of growth of a leukemia cell includes, for example, the (i) prevention of leukemic or metastatic growth, (ii) slowing down of the leukemic or metastatic growth, (iii) the total prevention of the growth process of the leukemia cell or the metastatic process, while leaving the cell intact and alive, (iv) interfering contact of cancer cells with the microenvironment, or (v) killing the leukemia cell.

Examples of anti-disease, anti-cancer, and anti-leukemic agents to which antibodies and fragments of the present invention may usefully be linked include toxins, radioisotopes, and pharmaceuticals.

Examples of toxins include gelonin, Pseudomonas exotoxin (PE), PE40, PE38, diphtheria toxin, ricin, or derivatives, combinations and modifications thereof.

Examples of radioisotopes include gamma-emitters, positron-emitters, and x-ray emitters that may be used for localization and/or therapy, and beta-emitters and alpha-emitters that may be used for therapy. The radioisotopes described previously as useful for diagnostics, prognostics and staging are also useful for therapeutics.

Non-limiting examples of anti-cancer or anti-leukemia agents include anthracyclines such as doxorubicin (adriamycin), daunorubicin (daunomycin), idarubicin, detorubicin, carminomycin, epirubicin, esorubicin, and morpholino and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof. Preferably, the anti-cancer or anti-leukemia is doxorubicin, morpholinodoxorubicin, or morpholinodaunorubicin.

In one embodiment, the present invention provides methods of inducing or activating ADCC by administering the present antibodies. Accordingly, these antibodies may activate ADCC and/or stimulate natural killer (NK) cells (e.g. CD56+), T-cytotoxic cells (e.g. CD8+), and/or monocytes, which may result in cell lysis. Generally, following administration of an antibody comprising an Fc region or portion of the antibody, said antibody binds to an Fc receptor (FcR) on effector cells, for example, NK cells, triggering the release of perforin and granzyme B, which then leads to apoptosis. Various factors can affect ADCC, including the type of effector cells involved, cytokines (IL-2 and G-CSF, for example), incubation time, the number of receptors present on the surface of the cells, and antibody affinity.

In one embodiment, the pharmaceutical compositions of the present invention have an antibody or fragment thereof with the binding capabilities of an scFv antibody fragment of SEQ ID NO: 1 and a pharmaceutically acceptable carrier. The antibody or fragment thereof can be present in an amount effective to inhibit or treat cell rolling, inflammation, infection, auto-immune disease, metastasis, growth and/or replication of tumor cells or leukemia cells, or increase in number of tumor cells in a patient having a tumor or leukemia cells in a patient having leukemia. Alternatively, the antibody or fragment thereof can be present in an amount effective to increase mortality of tumor cells or leukemia cells. Also alternatively, the antibody or fragment thereof can be present in an amount effective to alter the susceptibility of diseased cells to damage by anti-disease agents, tumor cells to damage by anti-cancer agents, or leukemia cells to damage by anti-leukemia agents. Further alternatively, the antibody or fragment thereof can be present in an amount effective to decrease number of tumor cells in a patient having a tumor or leukemia cells in a patient having leukemia.

Antibodies, constructs, conjugates, and fragments of the subject invention may be administered to patients in need thereof via any suitable method. Exemplary methods include intravenous, intramuscular, subcutaneous, topical, intratracheal, intrathecal, intraperitoneal, intralymphatic, nasal, sublingual, oral, rectal, vaginal, respiratory, buccal, intradermal, transdermal, or intrapleural administration.

For intravenous administration, the formulation preferably will be prepared so that the amount administered to the patient will be an effective amount from about 0.1 mg to about 1000 mg of the desired composition. More preferably, the amount administered will be in the range of about 1 mg to about 500 mg of the desired composition. The compositions of the invention are effective over a wide dosage range and depend on factors such as the particulars of the disease to be treated, the half-life of the peptide, or polypeptide-based pharmaceutical composition in the body of the patient, physical and chemical characteristics of an agent complexed with the antibody or fragment thereof and of the pharmaceutical composition, mode of administration of the pharmaceutical composition, particulars of the patient to be treated or diagnosed, as well as other parameters deemed important by the treating physician.

Pharmaceutical composition for oral administration may be in any suitable form. Examples include tablets, liquids, emulsions, suspensions, syrups, pills, caplets, and capsules. Methods of making pharmaceutical compositions are well known in the art (See, e.g., Remington, The Science and Practice of Pharmacy, Alfonso R. Gennaro (Ed.) Lippincott, Williams & Wilkins (pub)).

The pharmaceutical composition may also be formulated so as to facilitate timed, sustained, pulsed, or continuous release. The pharmaceutical composition may also be administered in a device, such as a timed, sustained, pulsed, or continuous release device. The pharmaceutical composition for topical administration can be in any suitable form, such as creams, ointments, lotions, patches, solutions, suspensions, lyophilizates, and gels. Compositions having antibodies, constructs, conjugates, and fragments of the subject invention may comprise conventional pharmaceutically acceptable diluents, excipients, carriers, and the like. Tablets, pills, caplets, and capsules may include conventional excipients such as lactose, starch, and magnesium stearate. Suppositories may include excipients such as waxes and glycerol. Injectable solutions comprise sterile pyrogen-free media such as saline, and may include buffering agents, stabilizing agents or preservatives. Conventional enteric coatings may also be used.

The antibody or fragment thereof and pharmaceutical compositions thereof, can be used in methods of treating a disease (e.g., treating can include ameliorating the effects of a disease, preventing a disease, or inhibiting the progress of a disease) in patients in need thereof. Such methods include inhibiting or treating cell rolling, inflammation, autoimmune disease, metastasis, growth and/or replication of tumor cells or leukemia cells, or increase in number of tumor cells in a patient having a tumor or leukemia cells in a patient having leukemia. In addition, such methods include increasing the mortality rate of tumor cells or leukemia cells or altering the susceptibility of diseased cells to damage by anti-disease agents, tumor cells to damage by anti-cancer agents, or leukemia cells to damage by anti-cancer agents. Such methods also include decreasing the number of tumor cells in a patient having tumor or leukemia cells in a patient having leukemia.

The present invention also provides a method of purging tumor cells from a patient by providing a sample containing cells from the patient and incubating the cells from the patient with an antibody of the present invention. In one embodiment, the purging occurs ex vivo.

### EXAMPLES

The following examples are set forth to aid in understanding and to further illustrate the invention, but are not intended and should not be construed to limit its scope in any way. Although specific reagents and reaction conditions are described, modifications can be made that are encompassed by the scope of the invention.

### Example 1

The present example demonstrates selection, production, and initial characterization of L32 scFv antibody fragments. Briefly, a phage display library displaying scFv antibody fragments was utilized to obtain and produce targeting molecules, and flow cytometry, particularly fluorescence-activated cell sorting (FACS), was used for identifying and isolating specific phage clones, the peptide or polypeptide of which recognizes target cells. The phage display library used herein was constructed from peripheral blood lymphocytes of 49 non-immunized human donors.

Phage clones were selected by and identified through a multi-step procedure known as biopanning. Biopanning was carried out by incubating phage displaying protein ligand variants (a phage display library) with target cells, removing unbound phage by a washing technique, and specifically eluting the bound phage. The eluted phage clones were optionally amplified before additional cycles of binding and optional amplification which enriched the pool of specific sequences in favor of those phage clones bearing antibody fragments which best bind to the target. After several rounds of panning, individual phage clones were characterized, and the sequences of the peptides displayed by the clones were determined by sequencing the corresponding DNA of the phage virion.

In the present invention, screening of T-lymphoma cells was carried out against non-defined epitopes for the initial biopanning steps, with subsequent clone selection performed with a desired target cell (e.g., B-leukemia cells, B-CLL cells, AML cells, multiple myeloma cells, and metastatic cells), the targeted cell surface markers of which are unknown.

Using the L1 protocol, the L32 scFv antibody clone was discovered by panning a phage display library on intact T-lymphoma cells. This protocol began with prewashing. One ml aliquots containing 2×10⁷ frozen leukemia/lymphoma T cells from patients, stored at −70° C., were quick-thawed at 37° C., and immediately diluted into 10 ml cold 2% PBS-Milk (MPBS). Cells were spun 5′ at 120×g at room temperature (RT), washed twice, suspended in MPBS, and counted with a hemocytometer. The scFv display phage library (Nissim et al., EMBO J., 13: 692-98 (1994)) was used with the agreement of the MRC. The library was originally constructed as a phagemid library displaying scFv fragments in which the V_{H} and the V_{L} domains were linked by a flexible polypeptide. The scFvs displayed in the phagemid library were fused to the N-terminus of the minor coat protein pIII of the phage, which was then subcloned into the pHEN1 vector. Repertoires of antibody fragments were first generated by PCR from rearranged V-genes of peripheral blood lymphocytes of unimmunized human (referred to as “naive repertoires”). To diversify the repertoire, random nucleotide sequences encoding heavy chain CDR3 lengths of 4-12 residues were introduced into a bank of 49 cloned human V_{H} gene segments. The fused V_{L} fragment in all the clones it derived from was a single unmutated V gene of germline IGLV3S1, creating a single pot library of approximately 10⁸ clones.

Selection of the L32 scFv antibody clone was carried out in a final volume of 0.5 ml MPBS containing 10⁶ T cells, 10¹¹ Colony Forming Units (CFU) of phagemids (Nissim library), and 10¹³ wild-type bacteriophage M13, with slow agitation for 1 hr at 4° C. Cell wash was then performed by suspending the cells with MPBS and centrifuging at 120×g at 4° C., as above. The selection+cell wash procedure was repeated three times.

After the first round of selection, bound phagemids were eluted from the T-lymphoma cells by incubating the cells for 5 mins at RT, with 150 μl of 0.1M glycine, pH 2.2. After neutralization, cells were spun and discarded, and the supernatant fluid containing the eluted phage particles was collected and designated E1 stock. This E1 stock was amplified by the addition of 1 ml of exponentially growing TG-1 cells and incubating for 30 mins at 37° C. An aliquot was plated for titration purposes, and the remaining volume was plated on large plates (150 mm) containing 2×TY/AMP (1.6% Tryptone, 1% yeast extract, 0.5% NaCl and 100 μg/ml ampicillin). Plates were incubated overnight at 30° C. To determine the output after each round of panning, colonies on the titration plate were enumerated, and the total output was calculated.

Fresh bacterial cultures of the bacterial strains TG-1 and HB2151 were prepared for infection (amplification) by growing the cells to A₆₀₀ of 0.5-0.9 (exponentially growing cells). E. coli TG-1 cells were used for phage propagation and E. coli HB2151 cells were used for scFv protein production. Colonies from the large plates were scraped and pooled. An aliquot (˜10⁷) of ampicillin resistant E. coli TG-1 cells was grown in liquid culture to A₆₀₀ of ˜0.5, then infected with helper phage (VSC-M13, Stratagene) to produce a large amplified phagemid stock. Phagemids were recovered by a PEG precipitation procedure (Harrison et al., Methods in Enzymology (1996) 267: 83-109). Approximately 10¹² phagemids/ml of amplified E1 stock, described above, was used for subsequent rounds of panning against T cells.

Second and third rounds of “sequential pannings” were carried out essentially as described for the first panning procedure with the following modifications: (i) for the second sequential panning, ˜10¹¹ phagemids were used, and (ii) following selection and wash, the bound phagemids were eluted by incubating the cells for 15 mins at RT, in 50 μl of PBS/1% BSA+ATP (10 mM). Cells were centrifuged and the supernatant fluid was collected. The output containing the eluted phagemids derived from the second round of this procedure, designated E1AT1, was used for the third round of sequential panning, as described above, without amplification. Following ATP elution, as in the second round, phagemids were amplified in TG-1 cells, as described above. The final amplified stock was designated E1AT2. One aliquot (5 μl) was mixed with TG-1 bacterial culture for infection, titration, and sequence analysis. The remaining volume (45 μl) was incubated with 1.3 ml TG-1 cell suspension for amplification and storage.

The estimated number of phagmids used for panning (input) and the estimated number of bound phagemids eluted (output) are summarized for the three consecutive steps of the L1 biopanning protocol in Table 2. The cell source and elution medium for each output result is listed, as well as the term used to distinguish each separate stock.

TABLE 2

| | | | | Amplified |
|---|---|---|---|---|
| Input stock | Cell source | Elution | Output | stock |
| Nissim library- | T- | Acid | 3.3 × 10⁶ | E1 |
| 10¹² | Lymphoma | | | |
| E1-10¹¹ | T- | ATP | 500 | E1AT1* |
| | Lymphoma | (10 mM) | | |
| E1AT1-500 | T- | ATP | 37 | E1AT2 |
| | Lymphoma | (10 mM) | | |

*Eluted but not amplified

The results shown in Table 1 indicate that, when ATP elution was used for the second and third rounds, the number of eluted phage was very low, indicating a possible increase in phage specificity.

Following panning, several clones from each round were selected for sequencing. The amino acid sequences presented in this section are as follows: (a) sequences which appeared more than once among the selected clones, (b) sequences of the CDR3 region of the heavy chain only (VH-CDR3), and (c) the species of VH germline of each isolated clone.

TABLE 3

| | | | | | |
|---|---|---|---|---|---|
| | VH-CDR3 | | | | |
| Clone | size | VH-CDR3 sequence | Germline | Output | Frequency |

| | | | | | | |
|---|---|---|---|---|---|---|
| L32 | 8 | Leu Asn Pro Lys Val Lys His | (SEQ ID NO:4) | VH3- | E1AT2 | 4/15 |
| | | Met | | DP32 | | |
| L31 | 7 | Leu Arg Gly Gly Asn Ala Met | (SEQ ID NO:5) | VH3- | E1AT2 | 5/15 |
| | | | | DP32 | | |

Two types of clones, L32 and L31, were identified following the L1 biopanning protocol, and their sequences are presented in Table 3. The number of amino acid residues in the CDR3 region (VH-CDR3 size) and the specific CDR3 sequences, together with the germline designation of each clone, are presented. In addition, the number of times that a specific clone type was isolated, as a function of the total number of clones sequenced for the L1 protocol (frequency), is presented. Interestingly, although the library contained VH from five different VH families (VH1, VH2, VH3, VH4, and VH6) with VH3 constituting ˜47% of the genes utilized, both isolated clones are of the VH3 family (DP32). This is an advantage for the scFv purification process, since Protein A Sepharose is used to purify scFvs derived from the VH3 family and cannot be used to purify clones derived from VH families other than VH3.

### Example 2

The present example describes production of various scFv antibodies used in comparison studies as controls.

The isolation and characterization of the Y1 and Y17 scFv antibody clones is described in detail in U.S. application Ser. Nos. 10/032,423; 10/032,037; 10/029,988; 10/029,926; 09/751,181; and 60/258,948 and International Application Nos. PCT/USO1/49442 and PCT/US01/49440.

In addition, negative control scFv clones were selected. For all binding experiments, a single clone was picked from the naive library (before selection). A phage stock and a soluble scFv, designated N14, were prepared from this clone. Sequence analysis indicates that it belongs to the V_{H}4-DP65 gene family. The sequence of the 11-mer V_{H}-CDR3 encoded by this clone, designated N14 CDR3, is SEQ ID NO:6. An additional negative clone, N01, was used in the binding analysis experiments. Clone N01 (reactive to recombinant hepatitis B virus [HBV] particles) belongs to the V_{H}3-DP35 family, and the sequence of the 9-mer V_{H}-CDR3 encoded by this clone, designated N01 CDR3, is SEQ ID NO:7.

The TM scFv antibody clones (below) were isolated using the TM protocol by panning a phage display library on T-lymphoma cell membranes. In this protocol, prewashing of T cells (2×10⁷) was performed as described above in the L1 protocol. Following prewashing of T cells, selection was carried out on the immobilized T-lymphoma cell membranes by adding 2 ml MPBS containing 10¹² phagemids from the original Nissim library. The tube was slowly agitated for 30 mins, then incubated for an extra 90 mins without agitation (both steps at RT). Excess unbound phagemids were removed by decanting the tube contents and washing the tube 10 times with PBS, 0.1% Tween, followed by 10 washes with PBS. For elution, exponentially growing E. coli TG-1 cells (2 ml) were added directly to the tube and incubated with slow agitation at 37° C. As above, an aliquot was plated for titration, and the remaining volume was plated for amplification. In addition, amplification was performed as described in the L1 protocol. The selection procedure was repeated for two additional rounds, using 10¹¹ phagemids of the previously amplified stock. The first clone of the amplified stock of the third panning procedure, on immobilized T cell membranes, was designated TM1.1-myc+/TM1.1. Several scFv antibodies were prepared from this clone, in particular TM1.1 and a variant thereof with a myc tag (TM1.1 -myc+).

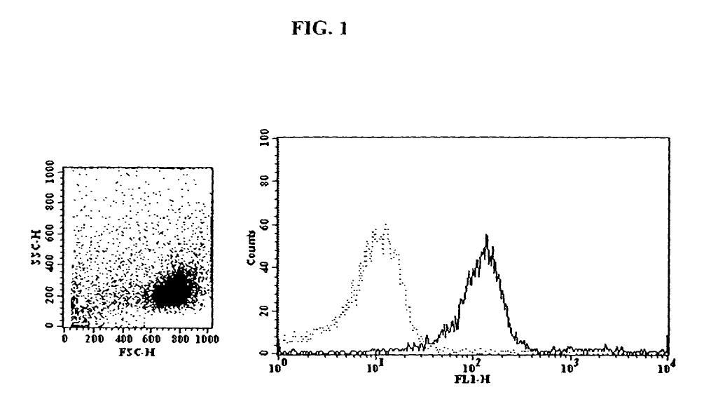

In addition to TM1.1-myc+ clone, the following additional clones were isolated by using the TM protocol. The amplified stock of the third membrane panning was used to pan intact T-lymphoma cells. The procedure was carried out essentially as described for the L1 protocol above, using 2×10⁷ cells and 10¹⁰ phagemids. Following 2 hrs of incubation at 4° C., bound phagemids were eluted from the washed cell pellet with 50 μl of Trypsin:EDTA (0.25%:0.05%), then neutralized by the addition of 50 μl of FCS. For titration and amplification, 1 ml of an E. coli TG-1 culture (A₆₀₀=0.5) was used. The amplified stock, designated TM2, was used for an additional round of panning on T-lymphoma cells, as above. The final stock was designated TM3. The sequences of scFv isolated as a result of following the TM protocol are presented in Table 4. Binding activity following FITC labeling of the scFv was also assessed to verify retention of scFv specificity (see Example 7). For example, the specificity of TM3.13 binding to T-ALL cells was verified by its binding according to FACS analysis (see FIG. 1).

TABLE 4

| | | | | | |
|---|---|---|---|---|---|
| | VH- | | | | |
| | CDR3 | | | | |
| Clone | size | VH-CDR3 sequence | Germline | Output | Frequency |

| | | | | | |
|---|---|---|---|---|---|
| TM2.3 | 7 | Leu Thr His Arg Ser Ser Arg | VH3- | TM2 | 2/10 |
| 1 | | | DP46 | | |
| TM2.2 | 7 | Thr Gln Arg Arg Asp Leu Gly | VH3- | TM2 | 5/10 |
| 3 | | | DP53 | | |
| TM3.2 | 7 | Lys Arg Val Ser Leu Leu Thr | VH3- | TM3 | 1/7 |
| 0 | | | DP70 | | |
| TM3.1 | 7 | Ser Tyr Arg Arg His Ser Arg | VH3- | TM3 | 2/7 |
| 8 | | | DP47 | | |
| TM3.1 | 11 | Arg Asp Lys Thr Thr Asn Phe Tyr | VH3- | TM3 | 1/7 |
| 3 | | Phe Met Lys | DP26 | | |

### Example 3

The present example demonstrates production, purification, labeling and characterization of L32 scFv clones

For production of soluble scFv, pHEN1, a vector used to construct the original phagemid library, was designed with an amber stop codon encoded at the junction of the scFv gene and the pIII gene. Therefore, when the vectors of selected clones are introduced by phagemid infection into E. coli HB2151, which is a non-suppressor strain, this system enables production and secretion of soluble scFv into the bacterial periplasm (Harrison et al., Methods in Enzymol. 267: 83-109 (1996)). The scFv is then readily retrievable from the culture broth. Soluble scFvs are produced under the control of the lacZ promoter (Gilbert and Muller-Hill, PNAS (US) 58: 2415 (1967)), which is then induced with IPTG (isopropylthiogalactoside).

A sequence encoding a c-myc tag of 10 amino acids—SEQ ID NO:8—is contained in the vector upstream to the amber mutation. The C-terminus of the expressed scFv should carry the c-myc tag, which can be detected using mouse anti-myc tag antibodies (derived from the European Collection of Cell Culture (ECACC) 9E10-hybridoma).

The scFvs of selected clones and of the control clone N01 all belong to the VH3 family, allowing purification on a Protein A affinity column. Periplasmic fractions (100-250 ml), from induced cultures of each clone, were prepared and incubated with Protein A Sepharose beads. The bound scFvs were recovered from the column by acid elution (0.1M glycine, pH 3.0), followed by eluate neutralization with Tris, pH.8.0. The concentration of the recovered protein was determined by A₂₈₀ measurement, followed by PBS buffer exchange by dialysis or on a G-25 Sepharose column.

The scFv of a clone L32 derived from the L1 protocol also belongs to the VH3 gene family (DP-32). However, following extraction from the periplasm, 5 mM DTT was required and added prior to loading the sample onto the Protein-A affinity column, after which purification on and recovery from Protein-A Sepharose beads and PBS buffer exchange was performed as described above.

The scFv of the negative clone N14 belongs to the VH4 gene family, which cannot be purified on a Protein A affinity column, therefore it was purified on a Sephacryl S-200 column. ScFv N14 was purified by precipitating the total protein in the periplasmic fraction of a 200 ml induced culture using 60% ammonium sulfate. The pellet was suspended in 2 ml 0.1×PBS, 5 mM EDTA, 5 mM PMSF and loaded on a Sephacryl S-200 column (1.5×90 cm) pre-equilibrated with the running buffer (0.1×PBS, 5 mM EDTA). Proteins were fractionated, and fractions containing the N14 scFv (as detected by SDS-PAGE and Western analysis) were pooled, lyophilized, and suspended in 1/10 volume H₂O. The N14 scFv (unlabeled and FITC-labeled) was then used as a negative control in FACS analysis experiments.

Purified scFvs were then labelled with FITC. Approximately 1 mg of purified scFv from each preparation was suspended in PBS and coupled to FITC using a Fluoro-Tag FITC conjugation commercial kit (Sigma-Aldrich Corp., St. Louis, Mo.), according to the manufacturer's instructions. Following purification and FITC labeling, the profile of each preparation (labeled and unlabeled) was analyzed by SDS-PAGE, Western blotting, HPLC using a Superdex-75 column (A₂₈₀ and A₄₉₅), and fluorometry. The analyses indicated 80% purity of the N14 scFv, and 90% purity for the VH3 clones, with approximately 2 molecules of FITC conjugated to each scFv molecule (F/P ratio of 2:1).

### Example 4

The present example demonstrates the binding of L32 scFv to washed platelets and platelet-rich-plasma (PRP) and the effects of L32 scFv on platelet aggregation.

For platelet aggregation studies, blood was collected into a tube containing 3.8% sodium citrate. PRP was prepared by centrifugation at 250×g for 10 mins. Platelet concentrate, in acid-citrate-dextrose (ACD), was obtained from a blood bank. Platelets were isolated, washed once with buffer containing ACD, and washed with saline in a ratio of 1:7. The platelets were centrifuged at 800 g for 10 min, after each wash, and were suspended in Tyrodes solution (2 mM MgCl₂, 137 mM NaCl, 2.68 mM KC1, 3 mM NaH₂PO₄, 0.1% glucose, 5 mM Hepes and 0.35% albumin, pH 7.35), and the number of cells was counted.

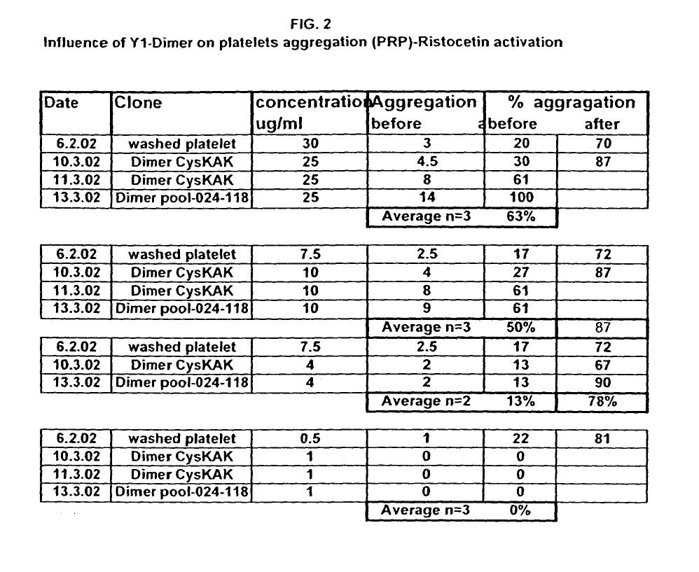

PRP and washed platelets were stirred at 500 rpm at 37° C., in whole blood, in a Lumiaggregometer (Chronolog, Havertown, Pa.). The difference in light transmission through the platelet suspension and suspending medium was taken as 100% aggregation. The effect of L32 on platelet aggregation was evaluated by addition of different concentration of L32 before the addition of agonist, and the effect was recorded for four mins (FIG. 2).

An aggregation assay was performed using washed platelets. The reaction mixture contains 2×10⁸ washed platelets/ml and 4 μg/ml of porcine von Willebrand factor. After a 3 min. incubation at 37° C., 0.4 mg/ml of ristocetin (an aggreggation agonist) was added, and the aggregation response was recorded for 4 mins. The effects of L32 and other scFv antibodies on platelet aggregation were evaluated by adding 50 μg/ml of the scFv before addition of the agonist, and the effect was recorded for 4 mins. The effect of L32 on vWF-dependent agglutination of washed platelets was tested in two samples of washed platelets (n=2), and normal agglutination (90% agglutination) was observed in contrast to abrogation of agglutination by Y1, a scFv with known effects. The effect of TM1.1-myc+, isolated using the TM protocol, was also assessed as a control. At the same conditions, the Y1 scFv antibody inhibited Ristocitin-induced platelet aggregation by ˜70% (FIG. 2).

For the aggregation assay using PRP, the reaction mixture contains PRP (˜2×10⁸ /ml). After 3 mins of incubation at 37° C., 1 mg/ml of ristocetin (an aggregation agonist) was added, and aggregation response was recorded for 4 mins. The difference in light transmission through the platelet suspension and PPP (plasma poor platelet) was taken as 100% aggregation. The effect of L32 on platelet aggregation was evaluated in PRP from three different donors (n=3) by adding 50 μg/ml L32 scFv before addition of the agonist, and the effect was recorded for 4 mins. Normal aggregation (90% aggregation) was observed after the addition of L32 scFv to PRP (FIG. 2). As mentioned above, the effect of L32 was compared with those of TM1.1 and Y1. Similar to washed platelets, Y1 inhibited PRP platelet aggregation by 70%.

In conclusion, 50 μg/ml of clone L32 had no significant inhibitory effect on platelet aggregation of washed platelets or PRP.

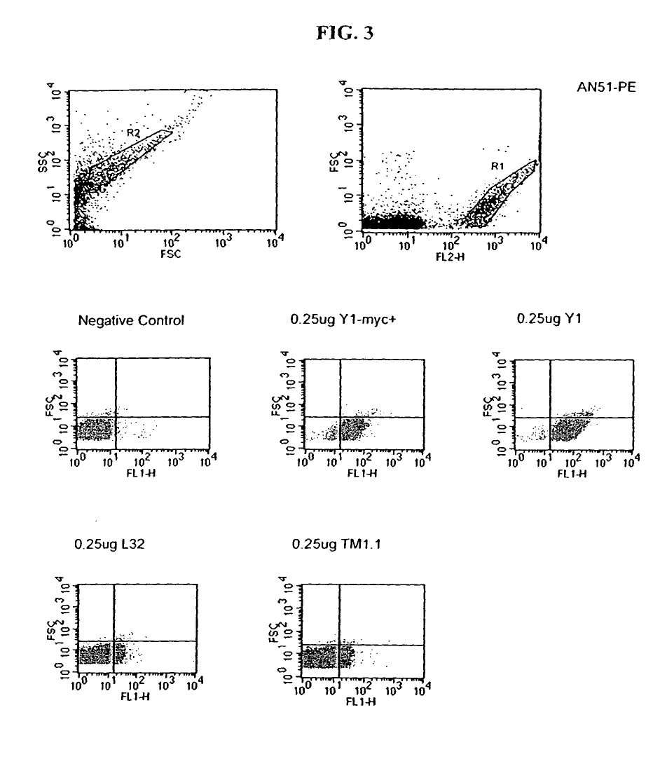

L32 scFv staining (binding) of platelets was also assessed by FACS. This method is useful for measurements based on the intensity of staining by fluorescent markers. As indicated in FIG. 3, staining with Y1 and Y1-myc+ scFv (a glycocalicin-reactive Y1 scFv antibody with a c-myc tag) resulted in stained platelets from PRP. In contrast, the fluorescent signal following staining of these platelets with L32 scFv was relatively unchanged, as compared with the staining with control antibodies (compare the histograms in FIG. 3, wherein TM1.1-myc+ is a scFv that does not bind to any platelet-associated epitopes).

### Example 5

The binding of scFv L32 to various different cell lines was analyzed by FACS. Analysis was carried out following three-step staining with (i) L32; (ii) anti-single chain antibody; and (iii) anti-Rabbit FITC-labeled antibody. The different cell lines were classified according to the ratio of the geo means of the cell population following L32 binding over that the negative control, as shown in Table 5. Low binding was assigned to cells with a ratio of 1, medium binding was assigned to cells with a ratio within the range of 1-4, and high binding was assigned to cells with a ratio greater than 4.

TABLE 5

| L32 | L32 | L32 |
|---|---|---|
| High | Medium | Low |
| KG-1 | Molt-4 | Raji |
| Jurkat | Hut78 | Daudi |
| | HEL | UMUC3 |
| | K562 | Namalwa |
| | CCRF- | |
| | CEM | |
| | HL60 | |

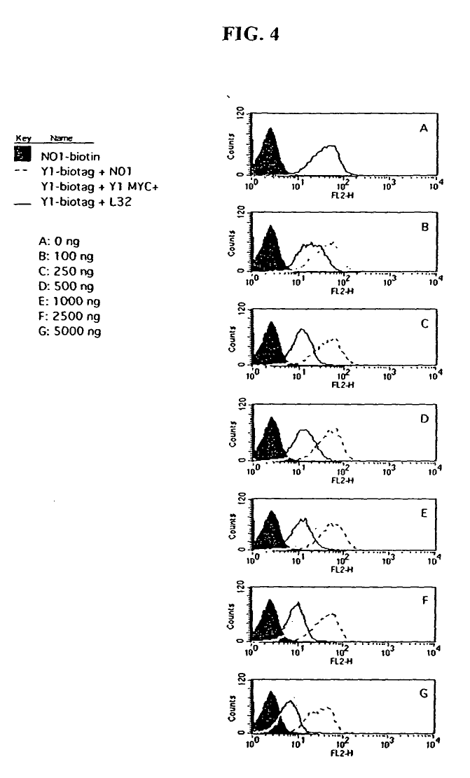

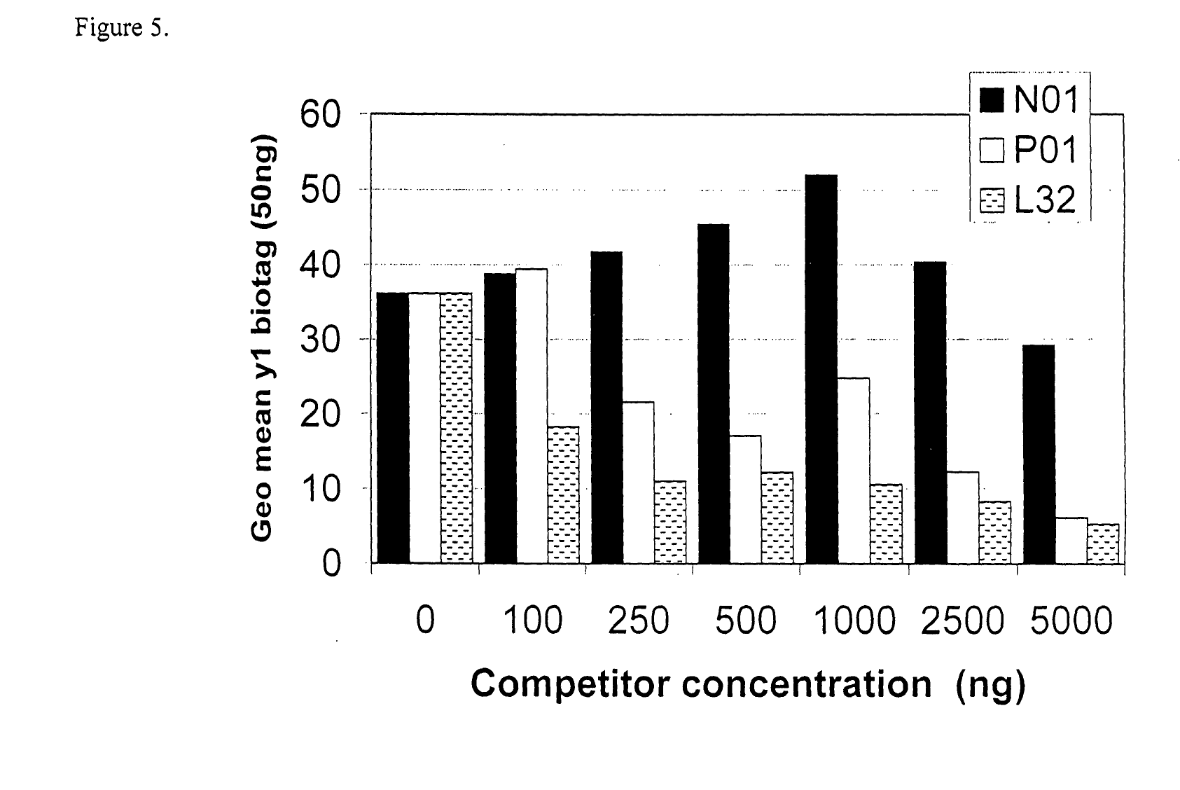

Competition for the same binding site between Y1 and L32 on these cells by these single chain antibodies was investigated. In one such experiment, competition of unlabeled antibodies for the biotin labeled Y1 (Y1-myc+) to KG-I cells (a human cell line derived from an AML patient) was assessed. The results are shown in FIGS. 4 and 5. As shown, L32 competes for Y1 binding on KG-1 cells similar to the Y1 scFv, itself. These results were supported by preliminary radioreceptor assay studies in which L32 scFv partially displaced the binding of ¹²⁵I-labeled Y1 scFv to KG-1 cells in a dose-dependent manner.

The capacity of unlabeled antibodies to compete with biotin-labeled L32 scFv (0.5, 2 or 5 μg) in binding to KG-1 cells was assessed in competition studies. Results presented in U.S. application Ser. Nos. 10/032,423; 10/032,037; 10/029,988; 10/029,926; 09/751,181; and 60/258,948 and International Application Nos. PCT/US01/49442 and PCT/US01/49440 demonstrate that Y1 and anti-CD162 antibodies compete for binding to CD162 antigen. FACS analysis was used to measure labeled antibody binding, and the results were expressed as the geometric mean values of binding. These results, as summarized in Table 6, demonstrate the concentration dependence of antibody displacement by Y1 scFv and anti-CD162 antibodies. When the amount of unlabeled antibody is much greater than the labeled antibody, more than 70% of the binding is displaced by the specific antibodies, while the nonspecific TM1.1 scFv has no significant effect on L32 binding. While anti-CD162 had the greatest capacity for displacing L32 (by as much as 89%), Y1 and L32 scFv antibodies displaced L32 binding at different degrees. Based on the results presented and others, less L32 is required than Y1 to compete for both L32 and Y1 binding, suggesting that L32 has greater binding capacity then that of the Y1 to the same site. Thus, these results further support the specificity of L32 binding and a substantial relation between the L32 epitope and those recognized by Y1 and anti-CD162 antibodies.

TABLE 6

| Inhibitor | L32 Biotin Concentration | | |
|---|---|---|---|
| Concentration | 0.5 μg L32-B | 2 μg L32-B | 5 μg L32-B |
| None | 22.65 | | |
| 50 μg TM1.1 | 21.6 | 28.3 | 38.32 |
| 50 μg Y1 | 6.36 | 12.99 | 21.99 |
| 50 μg L32 | 5.66 | 9.34 | 22.58 |
| 5 μg anti-CD162 | 4.06 | 3.95 | 4.4 |

Competition between L32, Y1, and anti-CD162 antibodies was further assessed by examining the degree of displacement of labeled anti-CD 162 antibody binding to KG-1 cells. Both L32 and Y1 scFv antibodies (50 μg) were found to reduce the geometric means of anti-CD162 (5 μg) labeling of KG-1 cells by approximately 82%. Thus, the epitopes of these antibodies are apparently closely related.

### Example 6

The present example demonstrates L32 scFv antibody binding to Glycocalicin (GC), a proteolytic fragment of GPIb, by ELISA.

GC was purified from fresh human platelets as described by Michalson (Blood 67: 19-26 (1986)). Before use in assays, the identity of GPIb was confirmed by EIA using two different commercial monoclonal antibody preparations; the first antibody (clone H1P1), purchased from Pharmigen (San Diego, Calif.), inhibits ristocetin-induced platelet aggregation, and the second antibody preparation (clone PM6/40), purchased from Serotec Inc. (Raleigh, N.C.), does not inhibit platelet aggregation.

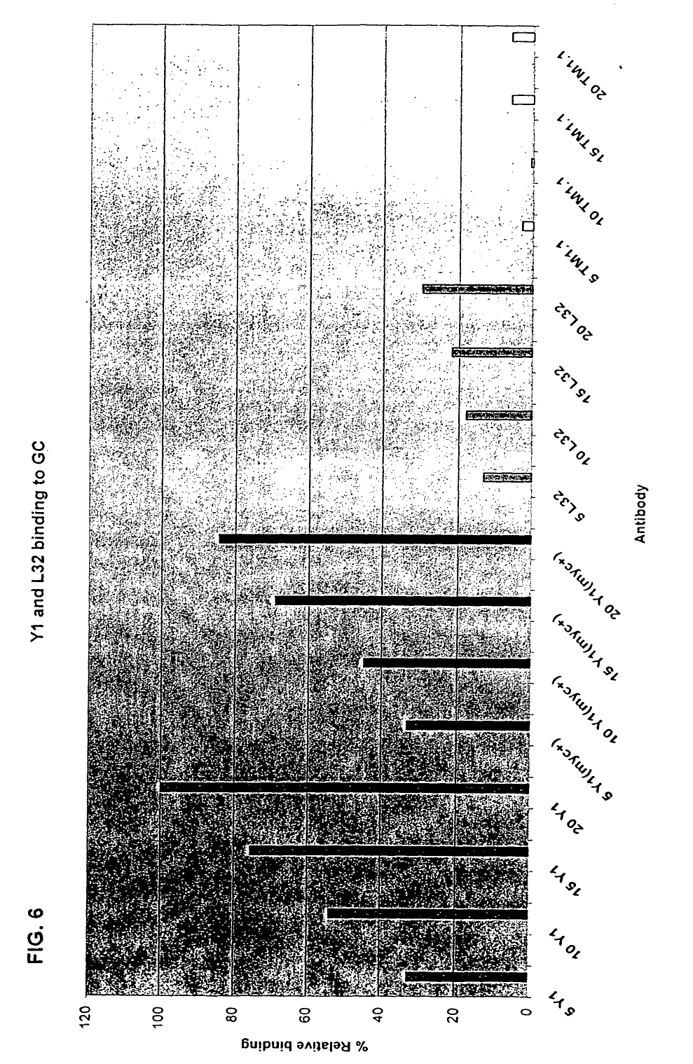

Quantitative analysis of binding of L32 to GC was determined by ELISA. GC diluted to 1 μg/ml (PBS X 1) was used to coat Maxisorp plate wells by overnight incubation at 4° C. After removing excess GC, the plates were blocked with PBSTM (phosphate buffered saline solution containing 2% milk and 0.05% Tween) at RT for 1 hr. After extensive washing with PBST, the plates were incubated with various concentrations of scFv diluted in PBSTM. Subsequently, the plates were incubated with anti-scFv diluted 1:250, or anti-VL 1:50, or anti-myc 1:100, followed by either anti-Rabbit HRP (Jackson) diluted 1:25,000 in PBSTM or anti mouse HRP 1:25,000 (as relevant). The reaction was developed with TMB and was stopped by the addition of 0.5M H₂SO₄. The plates were read at 450 nm using an ELISA reader, and the results of duplicate samples were averaged, with the ELISA unit values calculated by subtracting the background (staining without a primary antibody) OD value from the average values. The results depicted in FIG. 6 indicate that Y1 binding to GC is about 3-4 fold greater that that of L32.

### Example 7

The present example demonstrates L32 scFv antibody binding to proteins from various human derived sources by Western blot analysis.

Cell extracts (lysate) were prepared. Cells (2×10⁶) were harvested and centrifuged in a microcentrifuge (1300 rpm, 4° C., 5 mins). The pellet was washed with 0.5-1 ml PBS and gentle mixing, and the mixture was centrifuged as before. Washing with 0.5-1 ml PBS was repeated, and the pelleted cells were suspended in lysis buffer (200 μ/20×10⁶ cell pellet). The lysis buffer used was 50 mM Tris pH 7.4, 1 mm PMSF 1% NP-40, and 1 mM EDTA, although other suitable lysis buffers may be used. The suspension was incubated for about 60 mins, on ice, and then centrifuged (3000 rpm, 4° C., 5 mins). The supernatant was then collected and divided into aliquots.

A crude membrane fraction and extraction of membrane proteins were also prepared. Twenty volumes of homogenization buffer was added to one volume of packed cells. The homogenization buffer contained 2% (w/v) Tween 20, 1 mM MgSO₄, 2 mM CaCl₂, 150 mM NaCl, and 25 mM Tris-HCI, pH 7.4. The following protease inhibitors were also added: 1 mM PMSF, 5 μg/ml Leupeptin, and 5 μg/ml Aprotonin. The cells were homogenized using a Potter-Elvehjem homogenizer with a rotating Teflon pestle (Ultra-Torex) at a rate of three to five strokes. The sample was kept cold during homogenization, and then stirred for 1 hr in an ice bath. The sample was subjected to a few additional strokes in the homogenizer, and then centrifuged at 3000 g for 30 mins at 4° C. The supernatant was collected and centrifuged at 45000 g (19000 rpm rotor ss-34) for 1 hr at 4° C. The supernatant from the 45000 g centrifugation was discarded. A solution of 50 mM Tris 7.4, 1 mM EDTA, 1% NP-40 and protease inhibitors was added to the pellet, and the dissolved pellet was put on ice for 1 hr.

Plasma protein samples were prepared after diluting pooled plasma from a blood bank of healthy individuals 1: 10 (v/v) with PBS. The diluted solution was filtered through 0.45 μm membranes, and aliquots were stored frozen (−20° C.) until they were analyzed. Samples were then run on 10% SDS-PAGE at 140-160 Volts, for 3.5 hours at Sigma Z37, 503-9 appliance. The electrophoresed samples were transferred in Tris Glycine buffer (20% MeOH, 192 mM glycine, 25 mM TRIS, pH 8.3) onto a nitrocellulose membrane overnight at 20 Volts at RT.

The nitrocellulose membrane was blocked using 5% skim milk for one hr at RT. The membrane was then washed 3 times for 5 mins each with 0.05% Tween 20 in PBS at RT. The membrane was incubated with 5 μg/ml Y1-biotin, L32-biotin, or TM1.1-biotin in 2% skim milk, in PBS, for one hour at RT. The membrane was then washed 3 times for 5 mins each with cold 0.05% Tween 20, in PBS, in the cold room (about 4 to about 10° C.). The membrane was then incubated in the cold room with a 1:1000 dilution of SAV-HRP (streptavidin-HRP) (at a final concentration of 1 μg/ml), in 2% skim milk, 0.05% Tween. The dilution was carried out at RT (about 25° C.), and then the diluted SAV-HRP was cooled on ice for 10-15 mins before use. The incubation was carried out for 1 hour with gentle shaking.

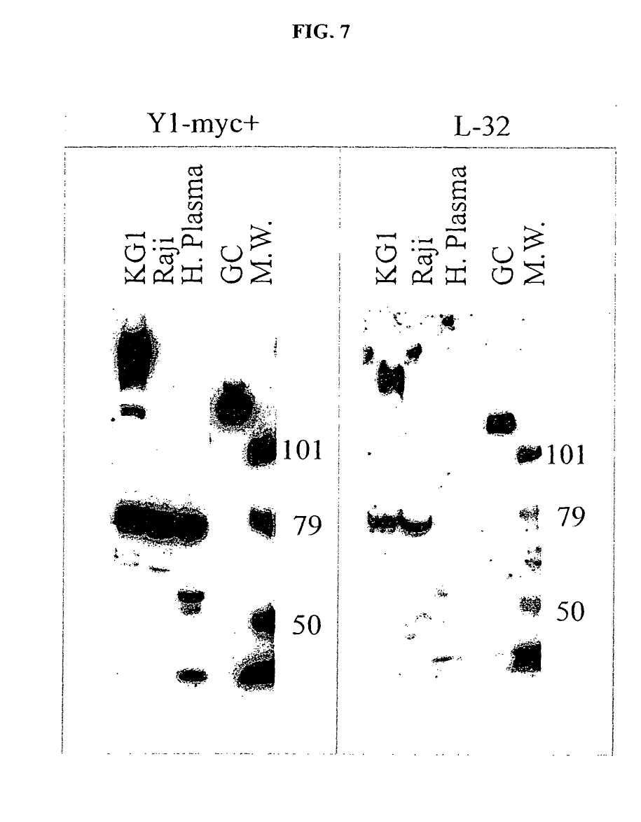

After the incubation with SAV-HRP, the membrane was washed, as above. The membrane was then incubated with Super Signal mixture (Pierce) for 5 mins as directed in the commercial protocol, then the excess solution was dried. The membrane was exposed to X-ray film (Fuji), and the film was developed. The results of these studies (FIG. 7) indicate that L32 binds to a protein on leukemia cells with a molecular weight of that of PSGL-1, approximately 105 kD. In addition, both L32 and Y1 react with the same bands of GC and KG-1 cells. However, L32 binding to plasma proteins is much lower than Y1, and Raji cell extract was negative to this >100 kDa band.

### Example 8

The present example demonstrates comparative binding to sulfated and non-sulfated peptides to L32 and Y1 by ELISA.

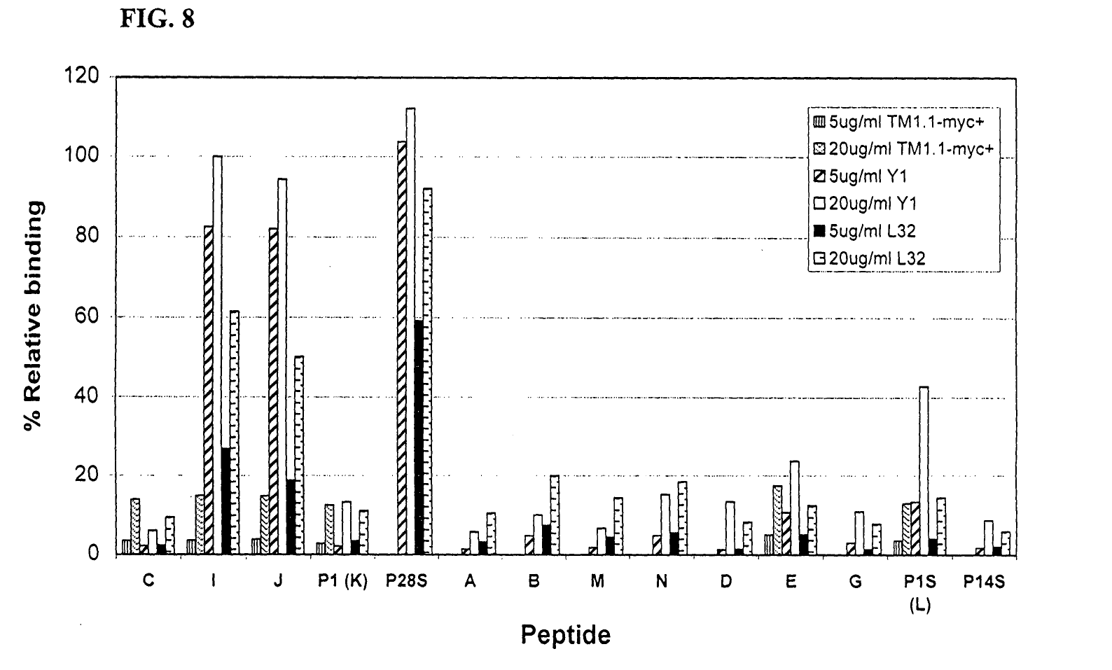

Sulfated and non-sulfated synthetic peptides based on the likely epitopes (amino acids 268 to 285 of GPIb and 1 -17 of mature PSGL-1) were prepared and used to assess the binding specificity of L32 scFv antibody (ELISA). Sulfated and nonsulfated peptides (1 μM) were attached to microtiter plates suitable for ELISA analysis, and unattached peptides were thoroughly washed before nonspecific binding sites were blocked. The plates were incubated with scFv antibodies in the indicated concentrations (see FIG. 8) for one hour at RT. The attached scFvs were detected using polyclonal rabbit anti-V_{L} antibodies, followed by horse raddish peroxidase (HRP) anti-rabbit antiserum. The samples were developed using the substrate TMB, and the peroxidase reaction was stopped, after approximately 10 mins, with 0.5M H₂SO₄. L32 binding to these peptides was compared with that of Y1 and TM1.1 (as a negative control) scFv antibodies. Background staining (in the absence of a primary antibody) was subtracted from each value to obtain the values presented in the bar graph. The peptides assessed for binding, and their essential structural properties are presented in Table 7.

TABLE 7

| | | | | | |
|---|---|---|---|---|---|
| Source of Peptide | Designation | Sequence | AA | MW | Sulfation |

| | | | | | |
|---|---|---|---|---|---|
| Fibrinogen γ prime | A | VRPEHPAETEYESLYPEDDL | 20 | 2389 | — |
| Fibrinogen γ prime | B | VRPEHPAETEY*ESLY*PEDDL | 20 | 2549 | Sulfated |
| PSGL-1-n-terminus | C | QATEYEYLDYDFLPETE | 17 | 2126 | — |
| PSGL-1-n-terminus | D | QATEY*EYLDYDFLPETE | 17 | 2206 | Sulfated |
| PSGL-1-n-terminus | E | QATEY*EY*LDYDFLPETE | 17 | 2286 | Sulfated |
| PSGL-1-n-terminus | F | QATEY*EYLDY*DFLPETE | 17 | 2286 | Sulfated |
| PSGL-1-n-terminus | G | QATEYEY*LDYDFLPETE | 17 | 2286 | Sulfated |
| PSGL-1-n-terminus | H | QATEYEY*LDY*DFLPETE | 17 | 2286 | Sulfated |
| PSGL-1-n-terminus | I | QATEYEYLDY*DFLPETE | 17 | 2286 | Sulfated |
| PSGL-1-n-terminus | J | QATEY*EY*LDY*DFLPETE | 17 | 2286 | Sulfated |
| GPIbα | K, P1 | GDEGDTDLYDYYPEEDTE | 18 | 2126 | — |
| GPIbα | L, P1S | GDEGDTDLY*DY*Y*PEEDTE | 18 | 2366 | Sulfated |
| GPIbα | P14S | GDEGDTDLYDYY*PEEDTE | 18 | 1732 | Sulfated |
| GPIbα | P28S | TDLY*DYYPEEDTE | 13 | 1732 | Sulfated |
| CCR5 | M | MDYQVSSPIYDINYYTSE | 19 | 2189 | — |
| CCR5 | N | MDY*QVSSPIY*DINY*YTSE | 18 | 2429 | Sulfated |

Y* designates sulfated Tyrosines.

Significant, dose-dependent L32 scFv antibody binding was obtained with peptides F, H, I and J, i.e. PSGL-1-related peptides sulfated at the third tyrosine residue, and with L and P28S, i.e., GPIbα-related peptides sulfated at the first tyrosine residue (FIG. 8). L32 scFv antibody binding to J was very similar to its binding to I. While binding to the GPIb-related L (P1S) peptide was significant, it was nonetheless comparably lower than that obtained with the PSGL-1- related peptides. Neither Y1 nor L32 were found to significantly bind the sulfated PSGL-1-related peptides G and D (lacking sulfation at the third residue). Furthermore, neither of Y1 and L32 bound sulfated (or non-sulfated) peptides related to the Fibrinogen γ-chain. Neither scFv antibody binds to the peptides in which the tyrosines are not sulfated, indicating that sulfation is required for binding. Moreover, data on the GPIb-related peptide, P28S, suggest that sulfation of the first tyrosine is significant for binding to GPIb, while data on the PSGL-1 peptides I and J, as compared to E, suggest that sulfation of the third tyrosine is significant for binding to PSGL-1. While Y1 and L32 display substantially identical patterns of binding behaviour with respect to the sulfated peptides (in this assay), Y1 appears to exhibit higher affinity binding relative to L32 in each case.

From the above experimental results and the data presented in U.S. application Ser. Nos. 10/032,423; 10/032,037; 10/029,988; 10/029,926; 09/751,181; and 60/258,948 and International Application Nos. PCT/US01/49442 and PCT/US01/49440, it was concluded that the epitope for the L32 scFv antibody is located between amino acids 1 and 17 on mature PSGL-1 in which there is cluster of negatively charged amino acids.

PSGL-1, which is a receptor for E, L- and P-selectins, was identified as a ligand of the Y1 antibody based on competition assays, wherein binding of the Y1 antibody to KG-1 cells was carried out in the presence of different commercially available anti PSGL-1 antibodies. The N-terminal region of PSGL-1 contains sulfated tyrosine residues accompanied by a cluster of negatively charged amino acids. (U.S. application Ser. Nos. 10/032,423; 10/032,037; 10/029,988; 10/029,926; 09/751,181; and 60/258,948 and International Application Nos. PCT/US01/49442 and PCT/US01/49440).

Although the Y1 antibody binds to several molecules, such as the glycocalicin molecule on platelets, fibrinogen-gamma prime, the complement compound 4 of human plasma, and the PSGL-1 molecule, its affinity to primary leukemia cells derived from either AML or multiple myeloma (MM) patients is higher relative to the previously mentioned epitopes.

While not wishing to be bound by any particular theory, the greater relative affinity of Y1 (as compared to that of L32) to sulfated peptides, as compared to the greater relative affinity of L32 (as compared to that of Y1) for leukemic cell lines and malignant cells, as shown in Examples 10, 16 and 17 below, may be due to factors such as possible differences in the conformation or exposure of the pertinent peptide sequences in the context of the full native proteins on cells. For example, the peptides used in the experiments were linear and did not have secondard and tertiary structure, as they would have in their native state. Such differences may not be evident in synthetic peptides, which are non-constrained linear peptides. Thus, the potential therapeutic application of L32 antibody binding may not be perceptible in the artificial synthetic peptide system, which is suggested by the cellular systems.

### Example 9

Results in U.S. application Ser. Nos. 10/032,423; 10/032,037; 10/029,988; 10/029,926; 09/751,181; and 60/258,948 and International Application Nos. PCT/US01/49442 and PCT/US01/49440 have demonstrated that Y1 and Y17 antibodies have similar recognition profiles on platelets. The present example demonstrates the effects of GPlb-derived peptide tyrosine sulfation and mutations on Y17 scFv antibody binding to washed platelets, and the dependence on tyrosine sulfation for Y17 binding to PSGL-1-derived peptides.

GPIb-derived peptides selected from Table 6 and two additional N-terminus-abbreviated GPIb-derived peptides with the following sequences were used in these studies: P2S —TDLY*DY*Y*PEEDTE and P25S—TDLYDY*Y*PEEDTE.

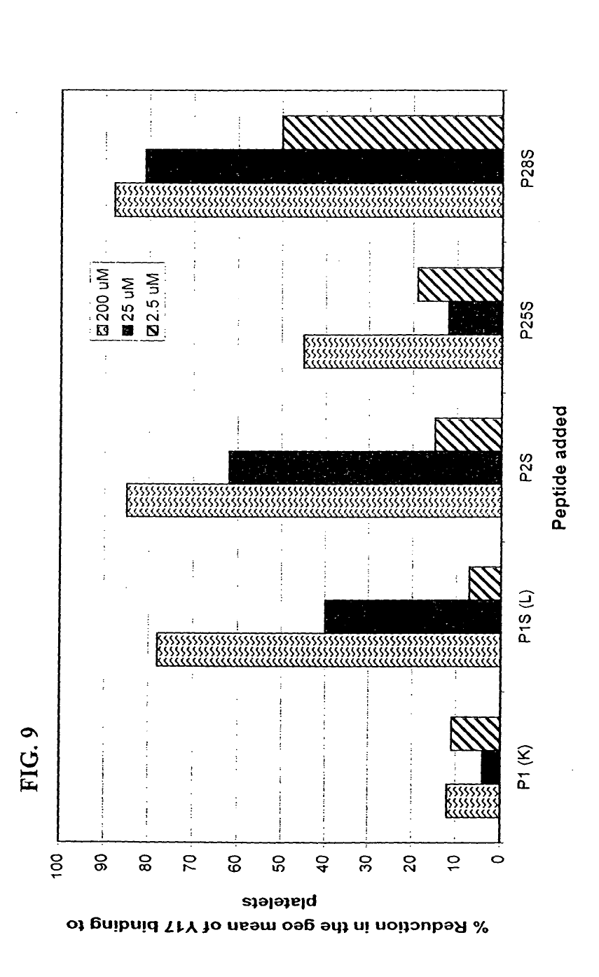

Y17 scFv antibody binding to washed platelets was examined by FACS as described in Example 4. The effects of the various peptides on Y17 binding to platelets were evaluated by first incubating Y17 together with the indicated concentration of peptide (see FIG. 9), before addition to the platelet preparation.

Of all of the peptides tested, GPIb-derived peptide containing sulfated tyrosine at position 276 (P28S) caused the greatest inhibition of Y17 binding to washed platelets. GPIb-derived peptides, containing amino acid changes were prepared and tested to confirm the consensus sequence requirements for Y17 recognition. These results suggest that the first sulfated tyrosine is important for Y17 binding to wash platelets. However, sulfation of the second Tyrosine apparently does not have a role in Y17 recognition. The negative charge amino acid residues of Aspartate (D) at positions 277 and 275 are also important for Y17 binding. The results were similar to those observed with Y1.

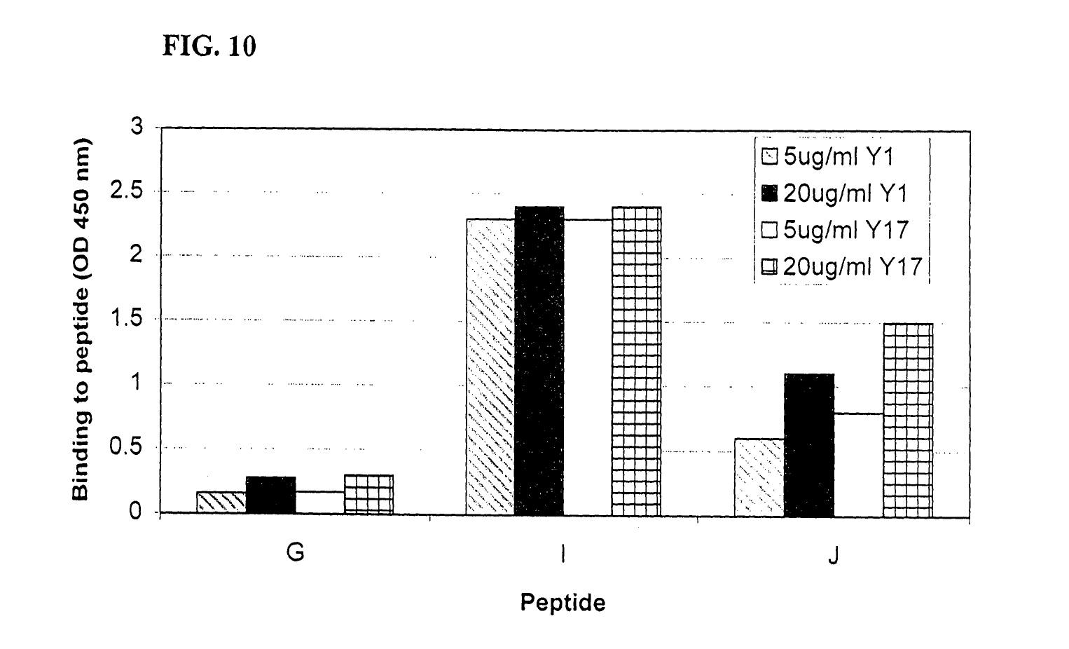

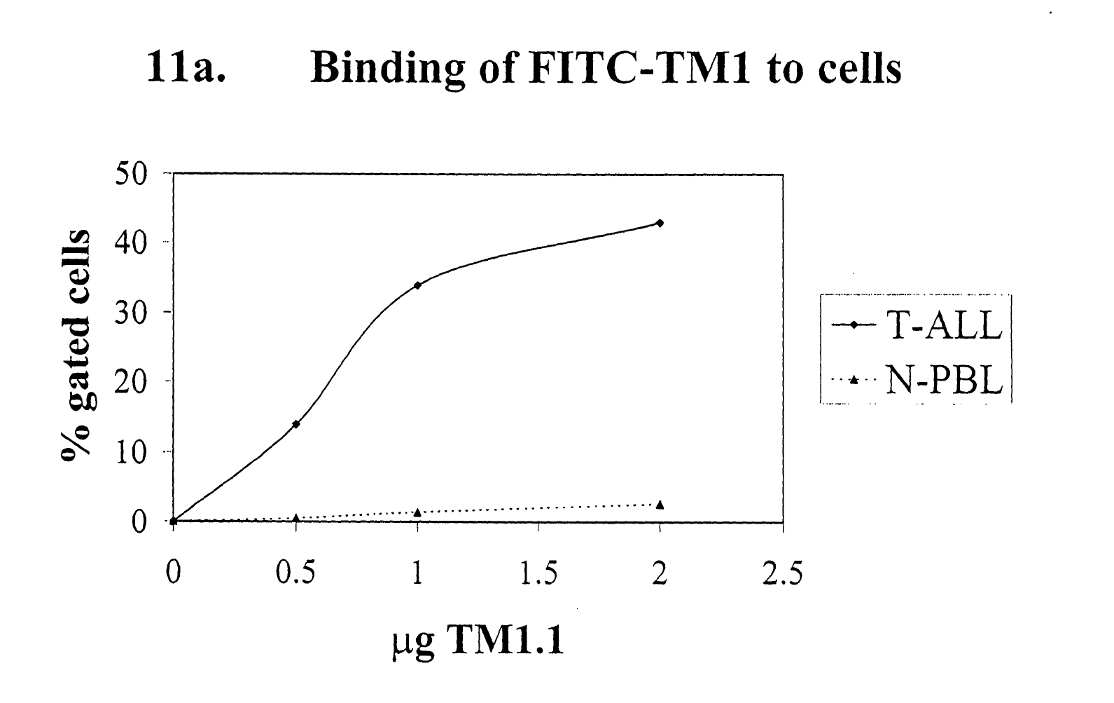

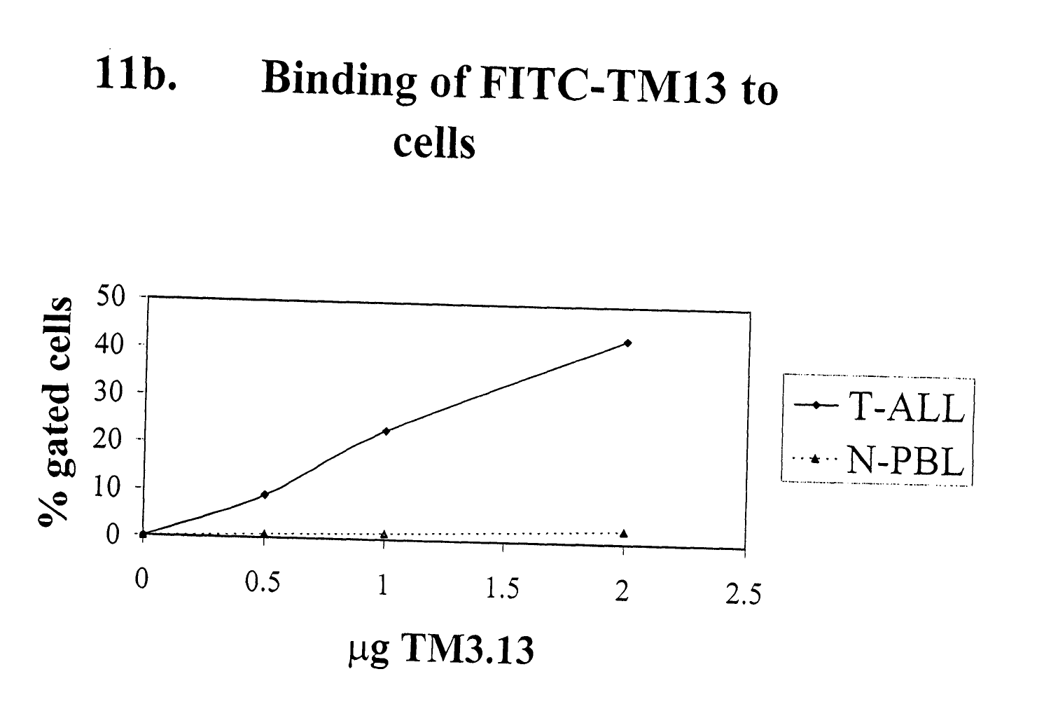

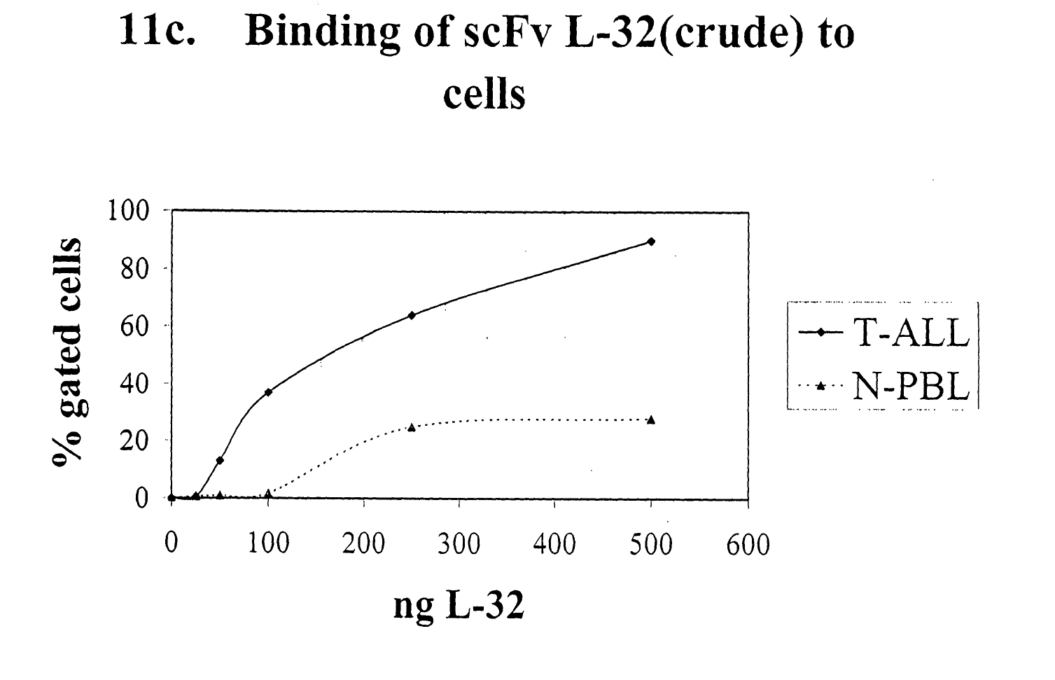

In order to verify what conditions determine Y17 scFv antibody binding to PSGL1, ELISA analysis was done using PSGL1-derived peptides bound to plates. Five and 20 μg/ml Y17 scFv antibody strongly bound to both PSGL1-derived peptides, I containing sulfated tyrosine at the third position and to J that contains three sulfated peptides. FIG. 10 indicates that Y17 scFv binding to J was slightly greater than Y1 binding to J, and both scFvs bound similarly to I. Their binding to I was greater than their binding to J. Neither Y1 nor Y17 significantly bound to G (PSGL1 peptide without sulfation).

In summary, at the peptide level, all three clones, Y1, Y17, and L32, have similar specificity profiles.

### Example 10

The present example demonstrates binding of L32 to primary cells from normal volunteers and leukemia patients.

All of the procedures for bacterial clone culturing, induction protocol, scFv antibody fragment harvesting, and antibody fragment purification were carried out in accordance with Harrison et al., (1996), supra. Basically, any two or more individual scFv clones can be selected from the Nissim I antibody phage display library in order to prepare rabbit derived polyclonal antibodies that recognize any individual scFv antibody present in the Nissim library or any IgG or fragment thereof provided that it contains the same V_{L} or a fragment thereof.

Polyclonal antibodies were raised against V_{L} derived from scFv antibody clone (Y1) derived from the Nissim I antibody phage display library. The DNA fragment encoding the V_{L} domain of human antibody was PCR-cloned from the Y1 clone (the identical DNA fragment can be obtained from any other clone in the Nissim I library (Nissim et al., (1994), supra) or even from the human genome using the same methodology) with the following synthetic oligonucleotide primers: oligo 5′-Nde1 (TTTCATATGGAGCTGACTCAGGACCCTGCT) and oligo 3′-EcoRI (TTTGAATTCCTATTTTGCTTTTGCGGC). After amplification by polymerase chain reaction (PCR conditions: 94° C. 1′, 56° C. 2′, 72° C. 2′×30 then 65° C. 5′), the obtained DNA fragment was digested with NdeI and EcoRI restriction enzymes and cloned into NdeI and EcoRI restriction enzyme sites of a pre-digested plasmid, which is an IPTG inducible expression vector used for prokaryotic expression of recombinant proteins in E. coli.

E. coli cells were transformed with the ligation mixture, and positive clones were selected by PCR amplification using the above oligonucleotide primers. Cells harboring this plasmid were grown and induced for expression by IPTG. Following induction with IPTG, bacterial cells were harvested by centrifugation after having been grown for 16 hours at 22° C., from 1 L of culture. Inclusion bodies were isolated and solubilized in guanidine-HCl+DTE and refolded by dilution in a buffer containing TRIS-ARGININE-EDTA. After refolding for 48 hrs at 5-10° C., the solution containing protein was dialyzed and concentrated in 20 mM Glycine, pH 9. The dialyzed solution containing proteins was re-purified using an ionic exchange column, HiTrapQ, and eluted with a gradient of NaCl. The main peak was analyzed by SDS-PAGE and by gel filtration. At least 10 mg of purified VL were obtained from the original 1-L culture.

Rabbits were then immunized with V_{L} (400 mg) in the presence of CFA (complete Fruend's adjuvant), then by V_{L} (200 mg) in the presence of IFA (incomplete Fruend's adjuvant) at 2 to 4 week intervals. The titers obtained were low (1:50-1:100), probably due to the high homology between the V_{L}s from human and rabbit.

Polyclonal anti-scFv antibodies were used, either directly from the serum of the immunized rabbits or after purification on a Protein A-Sepharose column, to detect scFv antibody binding to either cells analyzed by FACS or to various protein fractions separated by SDS-PAGE (Western blot analysis).

Generally, one of three FACS analyses was performed to test and confirm the specificity of the selected clones. A “three-step staining” procedure was established, using crude extracts or purified unlabeled scFv, followed by mouse anti-myc antibodies and, finally, FITC- or PE-conjugated anti-mouse antibodies. This procedure was alternatively done using rabbit anti-VL, as the second reagent, followed by FITC-labeled anti-rabbit antibodies. FACS analysis of cells, from blood or bone marrow samples, requires 5-8×10⁵ white blood cells, which have been suspended in PBS containing 1% BSA. Binding was carried out for 1 hr at 4° C. After each step, cells were washed and suspended in PBS containing 1% BSA. After the final staining step, lysis of red blood cells was the final step in the assay and was followed by suspending cells in PBS, then read by FACS (Becton-Dickinson). Analysis of cells stained by the “two-step staining” procedure was done as an alternative to the three-step procedure by first exposing cells to either myc- or biotin-labeled primary antibodies and subsequently staining them with either FITC-labeled anti-myc antibodies or PE-labeled strepavidin, respectively. A procedure for direct cell staining with scFv-FITC was established for subsequent FACS analyses. This new method requires only one incubation step for scFv -FITC labeling. In addition, the high intrinsic background, due to reactivity of the anti-myc with normal PBL, was much lower when scFv-FITC was used for FACS analysis. The results obtained with both direct FITC-scFv labeling and “three step staining” were very similar, indicating that the biological activity of the labeled scFv was not destroyed by the FITC labeling procedure. Thus, the labeled scFvs retain binding activity similar to that of the unlabeled antibodies.

The FACS protocol was carried out to analyze blood and bone marrow samples. Samples were provided from hospitals, with informed patient consent. Initially, cell samples were stained using the 3-step staining procedure in which detection of bound scFv antibodies was accomplished by mouse anti-myc tag antibodies followed by fluorescently labeled anti-mouse Ig antibodies. In this analysis, the results of which are presented in FIG. 11, the test scFvs (TM1.1, TM3.13, and L32 derived from T cell pannings) consistently exhibited high levels of binding to T-lymphoma/leukemia cells, as compared to normal peripheral blood lymphocytes (N-PBL) . In contrast, B-CLL cells exhibited relatively low levels of staining with the scFvs, similar to that exhibited by N-PBL (Table 8). When 1/50 scFv dilution was used for FACS analysis, only background binding was detected.

TABLE 8

| Cell type | Control | L32 |
|---|---|---|
| T- | 1.4 | 40 |
| lymphoma/leukemia | | |
| B-CLL | 0.2 | 5 |
| N-PBL** | 4.9 | 5 |

*Only anti-myc and anti-mouse-FITC were used
**Normal peripheral blood lymphocytes

In subsequent studies, patient whole blood or bone marrow samples (and samples from normal individuals) were adjusted to 30 μl/tube. Five microliters of CD33-APC (for AML) or CD19-APC (for B-CLL) or CD38-APC (for Multiple Myeloma) were added per tube, and 5 μl of CD45-PerCp and 5 μl of scFv Y1 or control scFv TM1.1 or CD162-PE (KPL1) were also added per tube. Tubes were incubated 30 mins at 4° C., with gentle shaking. Excess reagents were washed out by adding 2 ml PBS and spinning for 5 mins at 1200 rpm. The supernatant was discarded. In one-step assays, a lysis step was then performed. Five hundred microliters of BD Lysine solution was diluted 1:10 with ddH₂O, and 300 μl were added to each patient sample. The samples were vortexed at high speed, incubated for 12 mins at 4° C., and washed as above. After discarding the supernatant, 500 μl PBS were added. The samples were read with a FACS using blood sample acquisition setup according to international standards. For assays involving two or three steps, working buffer contained PBS+1%BSA+0.05% sodium azide, and incubations and washes were done as described above.

For blood samples from normal individuals, analysis was done to determine the selectivity of L32 binding to subpopulations of blood cells using FACS analysis as compared to Y1 selectivity. Binding was measured following staining with labeled second or third antibodies. There is between-donor variability in both Y1 and L32 binding. Accordingly, the results representing most of the cases are summarized qualitatively in Table 9. L32 scFv binding to granulocytes, lymphocytes, and monocytes was generally greater that Y1 scFv binding to these cells. In contrast, L32 scFv binding to platelets was similar to the background and generally less than that by Y1 scFv. These results further support those presented in Example 6 (see FIG. 2), which describe low effect on platelet aggregation in PRP and washed platelet agglutination.

TABLE 9

Binding Relative to Negative Control

| Cell type | Y1 | L32 |
|---|---|---|
| Lymphocytes | Background - Low | Low+/− |
| Monocytes | +/−Low | Medium+/++ |
| Granulocytes | Low - Medium+ | Medium - High++ |
| Platelets | +/−Low | Background− |

−background staining
+/−up to 2x the ratio of mean flow between negative scFv and tested scFv
+2x-3x the ratio of mean flow between negative scFv and tested scFv
++4x-6x the ratio of mean flow between negative scFv and tested scFv
+++6x-8x the ratio of mean flow between negative scFv and tested scFv
++++>10x the ratio of mean flow between negative scFv and tested scFv

Scaling is based on the following criteria (this is not an absolute scale—a 2× ratio might really be translated to several-fold higher binding):

It is important to note that all of the results in this table are derived using the two- or three-step staining procedure involving amplification of the signal. In addition, when more than one staining step is used, platelets are activated, and the signal resulting from a procedure-related effect is amplified. In such procedures, the labeled antibody binds to both GPIb and newly exposed PSGL-1 exposed on the activated platelets.

For blood/bone marrow samples from cancer patients, analysis was done to determine the selectivity of L32 binding using FACS analysis as compared to Y1 selectivity. Binding was measured as the result of binding by labeled second antibodies. The results, as presented in Table 10, indicate that L32 generally binds to diseased cells to a greater extent than does Y1. Concomitantly, L32 binding to granulocytes, lymphocytes, and monocytes was greater than that of Y1. This increased binding suggests reduced interactions with normal cells, which may translate into lower antibody doses for treatment.

TABLE 10

| | | Binding relative | |
|---|---|---|---|
| | | to negative control | |
| Disease | Cell type | Y1 | L32 |
| AML (4 | Disease | Ranged + to +++ | ++++ |
| samples) | Lymphocytes | Ranged + to ++ | +++ |
| | Granulocytes | +/− | ++ |
| MM (2 | Disease | +++ | ++++ |
| samples) | Lymphocytes | ++ | +++ |
| | Monocytes | ++ | +++ |
| | Granulocytes | ++ | +++ |
| B-Leukemia (2 samples) | Disease | Ranged − to ++ | + |
| | Lymphocytes | Ranged + to ++ | +++ |
| | Monocytes | +++ | ++++ |
| | Granulocytes | ++ | ++++ |

It should be noted that the commercial CD162-specific monoclonal antibody, anti-human PSGL1, displaced the binding of L32 in all tested samples, including AML, hairy cell leukemia, and B-cell malignancies (e.g. Pre-B-ALL, B-ALL, B-CLL, B-PLL, and multiple myeloma).

Full IgG, diabodies, triabodies, and Fab fragments all shared the specificity of the scFv antibodies, and anti-CD162 displaced binding of each antibody form.

### Example 11

The present example demonstrates binding of L32 to primary cells from animals of various species.

The selectivity of L32 binding to subpopulations of blood cells was assessed for blood samples from animals of various species. Whole blood samples were stained with either L32 or Y1 scFv, followed by PE-labeled rabbit anti-scFv (2-step staining). The samples were subsequently analyzed by FACS analysis. The results, as presented in Table 11, indicate that variation in binding by both antibodies exists between species. Nevertheless, Y1 generally stained granulocytes to a greater extent than L32, and Y1 was generally found to stain platelets, while L32 did not stain them, indicating that Y1, indeed, has broader specificity than L32.

TABLE 11

| | |
|---|---|
| | Binding According to |
| Species/Cell | FACS Analysis |

| population | Y1 | L32 |
|---|---|---|
| Mouse (Balb/c) | | |
| granulocytes | +/− | − |
| Platelets | ++ | − |
| other cell | − | − |
| populations | | |
| Rat | | |
| all cell populations | − | − |
| Rabbit | | |
| granulocytes | + | + |
| other cell | − | − |
| populations | | |
| Guinea pig | | |
| lymphocyte | + | − |
| Monocyte | ++ | − |
| granulocyte | ++ | − |
| Platelet | + | − |
| Dog | | |
| lymphocytes | − | − |
| monocytes | ++ | +/− |
| granulocytes | + | − |
| Platelets | +++ | − |

### Example 12

The present example demonstrates the effects of administration of L32 to mice bearing malignant cells of human origin.

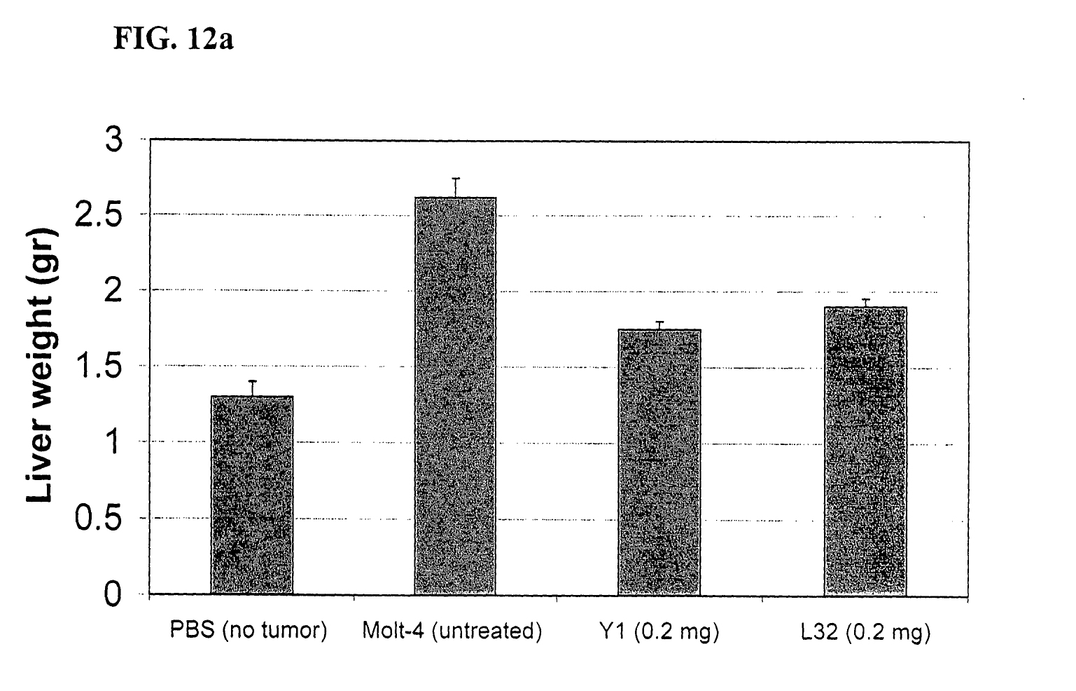

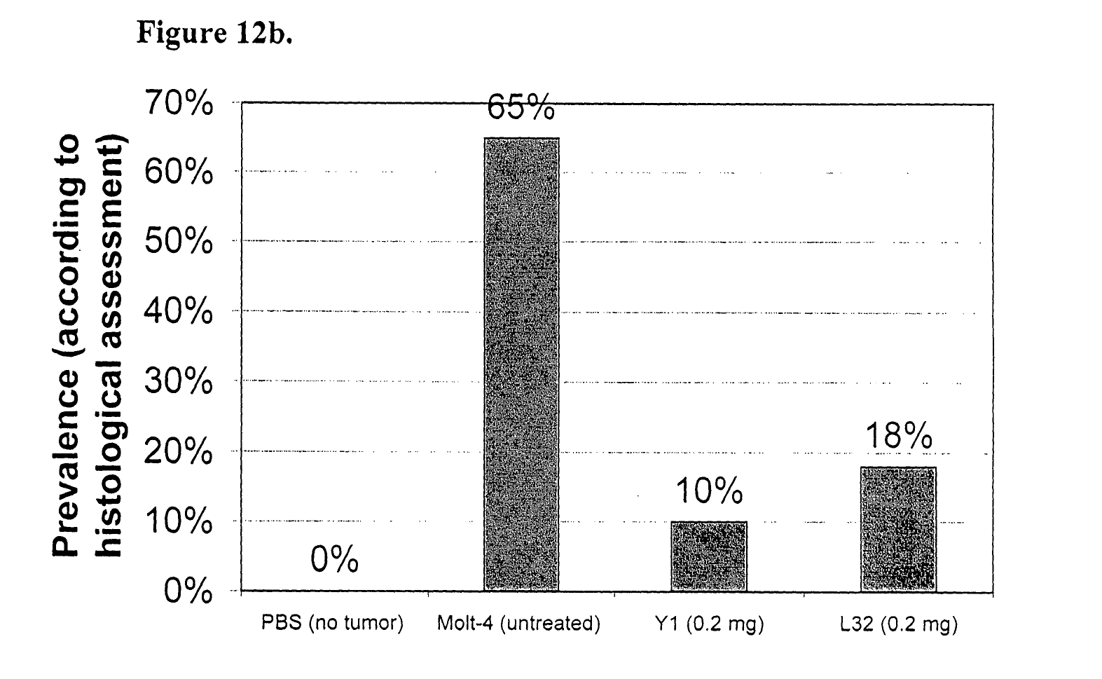

SCID mice (Jackson) were pretreated with 100 mg/kg CTX, and Molt-4 (T leukemia) cells were inoculated intravenously (i.v.) through the tail vein 5 days after CTX injection. Mice were randomly divided into treatment groups (6 per group), and they were treated 5 days later, for three weeks, with PBS, Molt-4, Y1 and two weeks with L32. The mice of the Molt-4 group were not treated. On Day 33, the mice were sacrificed, and their livers weighed. FIG. 12 demonstrates that the liver weight of mice with Molt-4 growths was doubled, and the tumor prevalence as measured by histology was ˜65%. Treatment with either Y1 or L32 scFv antibodies substantially reduced the tumor load of treated mice.

### Example 13

The present example demonstrates construction, expression and purification of L32 diabodies and triabodies.

The vector pHEN-L32 encoding the original L32 is amplified using PCR for both the V_{L} and the V_{H} regions, individually. The sense oligonucleotide and the anti-sense oligonucleotide are used for the V_{L} PCR reaction. The cDNA product with the expected size is purified, sequenced, and digested with restriction enzymes. The same procedure is employed to amplify the V_{H} region. The VH PCR product is digested with restriction enzymes. A triple ligation procedure into the pHEN vector, pre-digested, is employed. The final vector is designated pTria-L32. Following E. coli transformation, several clones are picked for further analysis, which includes DNA sequencing, protein expression, and extraction from the periplasmic space of the bacteria. SDS-PAGE under reducing conditions and Western blot analysis are performed to confirm the size of the L32 triabodies.

The pTria-L32 vector is linearized with a restriction enzyme, and synthetic complimentary double stranded oligonucleotides are pre-annealed and ligated into the restriction site between the L32-heavy and L32-light chains. This new vector is designated pDia-L32. As described for the triabodies, the DNA sequence and protein expression are confirmed.

Expression in E coli is essentially as described for the scFv L32. However, the purification of L32 diabodies and triabodies from the periplasm of the transformed E. coli cells is different. The scFv L32 monomer form can be purified on an affinity column of Protein-A Sepharose beads. Multimeric forms of L32 are, however, ineffectually purified by this procedure, therefore periplasmic proteins extracted from the bacteria are precipitated overnight with 60% ammonium sulfate, suspended in H₂O, and loaded onto a Sephacryl-200 (Pharmacia) size exclusion column pre-equilibrated with 0.1.×PBS. Fractions are collected and analyzed by HPLC, and separate fractions containing either the dimer or timer forms are collected for FITC labeling and FACS analysis.

### Example 14

The present example shows production of L32-cys-kak (cysteine dimer).

One liter of λpL-L32-cys-kak bacterial culture was induced for 2-3 hrs at 42° C. This culture was centrifuged at 5000 RPM for 30 mins and the pellet suspended in 180 ml of TE (50 mM Tris-HCl pH 7.4, 20 mM EDTA), and 8 ml of lysozyme (from a 5 mg/ml stock) was added and incubated for 1 hr. Twenty ml of 5M NaCl and 25 ml of 25% Triton were added and incubated for another hour. This mixture was centrifuged at 13000 RPM for 60 mins at 4° C. and the supernatant discarded. The pellet was suspended in TE with the aid of a tissuemiser (or homogenizer). This process was repeated 3 to 4 times until the inclusion bodies (pellet) are gray/light brown in color.

The inclusion bodies were solubilized in 6M Guanidine-HCl, 0.1M Tris (pH 7.4), 2 mM EDTA (1.5 grams of inclusion bodies in 10 ml solubilization buffer provided ˜10 mg/ml soluble protein). This was incubated for at least 4 hrs. The protein concentration was measured and brought to a concentration of 10 mg/ml. DTE was added to a final concentration of 65 mM and incubated overnight at RT. Re-folding was initiated by dilution of 10 ml of protein (drop by drop) to a solution containing 0.5 M Arginine, 0.1 M Tris pH 8, 2 mM EDTA, 0.9 mM GSSG. The re-folding solution was incubated for 48 hrs at ˜10° C. The re-folding solution containing the protein was dialyzed in a buffer containing 25 mM Phosphate buffer pH 6, 100 mM Urea, and concentrated to 500 ml. The concentrated/dialyzed solution was bound to an SP-sepharose column, and the protein was eluted by a gradient of NaCl (up to 1M).

### Example 15

Production of full L32 IgG antibody and Fab₁ and F(ab′)₂ fragments is to be performed as follows, as has been done for Y1 IgG.

CHO^{—} cells are cultivated in F-12 medium supplemented with 10% fetal calf serum and 40 μg/ml gentamicin at 37° C., in 5% CO₂ atmosphere. One day before transfection, 1-1.5-1×10⁶ cells are seeded on 90 mm dishes. The cultures are co-transfected with 10 μg of DNA encoding for the light and heavy chains of the L32 antibody. Transfection is carried out with the FuGene (Roche) transfection reagent technique. After 2 days of growth in nonselective growth media, the cells are cultured for 10-12 days in F-12 medium containing 550 μg/ml neomycin and 3 μg/ml puromycin. The cells are trypsinized and cloned by limiting dilution of 0.5 cell/well in Costar 96-well plastic plates. Individual colonies are picked, grown in six-well dishes and transferred to flasks for further selection (to determine level of expression and antibody secretion to the growth media).

CHO^{—} cells are cultivated in F-12 medium supplemented with 10% fetal calf serum and 40 μg/ml gentamicin at 37° C. in 5% CO₂ atmosphere. One day before transfection 0.8-1×10⁶ cells are seeded on 90mm dishes. The cultures are transfected with 10 μg of DNA encoding for the light and heavy chains of the L32 antibody cloned under the CMV (cytomegalo virus) promoter and the dhfr gene under the sv-40 promoter. Transfection is carried out using the FuGene (Roche) transfection reagent technique. After 2 days of growth in nonselective growth media, the cells are cultured in a media containing 100 nM-5 μM of methotrexate (MTX) and dialyzed fetal calf serum in order to select for clones (after limiting dilution) that express increased levels of the full L32 antibody.

A sandwich ELISA assay is established to determine the antibody concentration that is secreted into the supernatant of transfected CHO cells. In order to quantify the antibody concentration, the following reagents are used: a monoclonal anti-human IgG1 (Fc) (Sigma) as the coated antibody, a goat anti-human IgG (γ-chain specific) biotin conjugate as the detector (Sigma), and a purified human IgG1, lambda (Sigma) as standard.

Cells are grown in roller bottles to a final concentration of 1-2×10⁸ cells per bottle in F-12 medium supplemented with 10% fetal calf serum, neomycin and puromycin (as indicated above). For antibody production, cells are cultured in the same medium, but with 2% of fetal calf serum for additional two days. The secreted antibody is purified on a protein G sepharose column (Pharmacia) and ion exchange column-Q sepharose (Pharmacia). Binding is in 20 mM sodium phosphate buffer pH 7.0, while elution is in 0.1M glycine buffer, pH 2.5-3.0. The quantity of the purified antibody is determined by UV absorbance and ELISA, while its purity is analyzed by SDS-PAGE and HPLC.

Fragmentation of L32 IgG into Fab₁ and F(ab′)₂. Initially, monovalent and divalent antibody fragments are prepared by using immobilized Ficin (Pierce). Ficin cleavage produces F(ab′)₂ fragments in the presence of 1 mM cysteine. Similarly, by increasing the concentration of cysteine activator in the digestion buffer to 10 mM, Fab fragments can be created from the original IgG. After digestion, the fragments are purified on an immobilized Protein A column. The F(ab′)₂ and Fab₁ fragments are concentrated using a microconcentrator with either a 10,000 or 30,000 Dalton molecular weight cutoff. Protein recovery is determined using absorbance at 280 nm. Fragment purity is determined using gel electrophoresis.

Further, 10 mg of purified L32 antibody is applied to a 0.5 ml of immobilized Papain slurry in digestion buffer at 37° C., for 16 hrs. Reaction is terminated by eluting the digest with 1.5 ml of binding buffer. Separation of Fab₁ from undigested IgG and Fc fragments is done using Protein A column with Binding buffer. The Fab₁ is contained in the flow through. By reading the absorbance at 280 nm, the peak fractions containing the fragments are pooled, concentrated and dialyzed against PBS, pH 7.4, overnight. Protein recovery, purity and characterization is determined by absorbance at 280 nm and gel electrophoresis.

### Example 16

The effect of scFv L32 on the binding of scFv Y1 (Table 11) or IgG Y1 (Table 12) antibodies to ML2 cells was assessed by competition assays using FACS analysis.

One microgram of competing antibody (L32 scFv, Y1 scFv (P03), KPL-1 or control N06 scFv) was added to ML2 cells (0.5×10⁶ cells per assay). After 30 minutes of incubation, Y1-PE labeled scFv or IgG was added for an additional 30 minutes of incubation at 37° C. Post incubation the samples were washed once with FACS buffer and analyzed.

The results are given in Geo mean number as a median average of two duplicate tubes analyzed. Geo mean value is an exponential and not linear expression of binding affinity.

The results shown in Table 12 indicate that L32 scFv effectively inhibits (by about 80%) binding of scFv Y1-PE to ML2 cells, i.e., L32 scFv inhibits in a manner similar to Y1 scFv. In contrast, the non-related negative control antibody scFv N06 inhibits the binding by only 20%.

TABLE 12

| Sample | Median | Percent Inhibition |
|---|---|---|
| Control | 5 | — |
| KPL-1-PE | 700 | — |
| Y1-PE | 240 | — |
| Y1-PE + N06 | 190 | 20 |
| Y1-PE + P03 | 48 | 80 |
| Y1-PE + L32 | 40 | 83 |

TABLE 13

| | Median | | | Percent Inhibition | | |
|---|---|---|---|---|---|---|
| Sample | 200 ng | 500 ng | 1000 ng | 200 ng | 500 ng | 1000 ng |
| Control | 3 | ND | ND | — | ND | ND |
| Y1 | 12.5 | ND | ND | — | ND | ND |
| Y1-PE + Y1 | 3 | ND | ND | 100% | ND | ND |
| Y1-PE + | 2 | ND | ND | 100% | ND | ND |
| KPL-1 | | | | | | |
| Y1-PE + P03 | 7.5 | 6 | 5 | 40% | 52% | 60% |
| Y1-PE + L32 | 5.5 | 3.8 | 3.7 | 54% | 79% | 80% |

ND = not done since complete inhibition achieved at lower concentration.

The results shown in Table 13 indicate that while IgG Y1 alone (final concentration 200 ng) bound to the cells with a Geo Mean of 12.5, competition with 200 ng of either “cold” IgG Y1 or KPL-1 (commercially available murine monoclonal antibody against PSGL1) reduced the Geo Means of IgG Y1 binding to 3 and 2 respectively. The binding of IgG Y 1 to ML2 cells was inhibited (competed) by the scFv Y1 antibody in a dose dependent manner, i.e., 200 ng of scFv Y1 antibody reduced the binding of IgG Y1 to a Geo Mean of 7.5 while 1000 ng reduced it to a Geo Mean of 5. The binding of IgG Y1 to ML2 cells was also inhibited (competed) by the scFv L32 antibody in a dose dependent manner, but to an even greater extent than that exerted by scFv Y1, i.e., 200 ng of scFv L32 antibody reduced the binding of IgG Y1 to a Geo Mean of 5.5 while 1000 ng reduced it to a Geo Mean of 3.7.

While both scFv Y1 and scFv L32 inhibited the binding of IgG Y1 to ML2 cells, at identical antibody concentrations, inhibition exerted by scFv L32 is more pronounced than that exerted by scFv Y1. Moreover, both IgG antibodies tested (IgG Y1 and KPL-1) have higher affinity to the ML2 cells relative to the two scFv antibodies tested as they reduce the Geo Mean of IgG Y1 binding to a Geo Mean of 3 and 2 respectively.

### EXAMPLE 17

The present example demonstrates binding of L32 to various subpopulations of blood cells, including both normal (Table 14) and diseased cells (Table 15), using FACS analysis.

First, analysis was carried out to determine the selectivity of L32 scFv binding to CD34+ precursor cells, isolated from normal human bone marrow (NBM) samples, as compared to Y1 scFv. Binding was measured following staining with labeled antibodies (anti-scFv—PE). Each NBM sample was tested with both antibodies in identical assays.

The criteria used to evaluate binding are as follows:

Geo Mean (GM) below 10—Negative

Geo Mean (GM) between 11 and 20—Low Affinity Binding

Geo Mean (GM) between 21 and 40—Medium Affinity Binding

Geo Mean (GM) between 41 and above—High Affinity Binding

As shown in Table 14, of 32 NBM samples tested, 24 (75%) did not bind either L32 or Y1. Six samples (19%) showed low affinity binding for L32, 1(3%) showed medium affinity binding for L32, and 1 (3%) showed medium affinity binding for both L32 and Y1. Overall, the binding profiles of L32 and Y1 to CD34+ cells were similar.

TABLE 14

| Number | Code | P03 | L32 |
|---|---|---|---|
| N1 | 105351 | neg. | neg. |
| N2 | AH/NBM01 | neg. | 13 |
| N3 | AH/NBM03 | neg. | neg. |
| N4 | AH/NBM04 | neg. | neg. |
| N5 | KBM33 | neg. | 14 |
| N6 | AH/NBM08 | neg. | neg. |
| N7 | AH/NBM09 | 10 | 13 |
| N8 | AH/NBM10 | 10 | 14 |
| N9 | AH/NBM11 | neg. | neg. |
| N10 | AH/NBM12 | neg. | neg. |
| N11 | AH/NBM13 | neg. | neg. |
| N12 | 105377 | neg. | neg. |
| N13 | AH/NBM14 | neg. | neg. |
| N14 | AH/NBM20 | 9 | 16 |
| N15 | AH/NBM22 | neg. | neg. |
| N16 | AH/NBM23 | neg. | neg. |
| N17 | AHSBM24 | neg. | neg. |
| N18 | AHJBM26 | neg. | neg. |
| N19 | AHSBM27 | 9.6 | 22 |
| N20 | AHSBM28 | neg. | 17 |
| N21 | AHJBM31 | 26 | 28 |
| N22 | AHJBM36 | neg. | neg. |
| N23 | AHJBM37 | neg. | neg. |
| N24 | AHJBM38 | neg. | neg. |
| N25 | AHJBM39 | neg. | neg. |
| N26 | AHJBM40 | neg. | neg. |
| N27 | AHJBM41 | neg. | neg. |
| N28 | AHJBM42 | neg. | neg. |
| N29 | AHJBM43 | neg. | neg. |
| N30 | AHJBM44 | neg. | neg. |
| N31 | AHJBM45 | neg. | neg. |
| N32 | 102546 | neg. | neg. |

The numbers in the table represent the geometric mean of the sample

In addition, comparative FACS analysis of binding of L32 scFv and Y1 scFv (PO3) to human primary leukemia cells (AML, MM, B-CLL, and B-ALL) isolated from patient's blood samples was carried out. The binding of L32 was compared to that of the Y1 in the same samples in identical assays. The binding criteria are as set forth immediately above.

Table 15 shows the data obtained from all of the patient samples tested, and Table 16 summarizes the affinity data for L32 and Y1 affinity in AML and B-CLL samples.

TABLE 15

| | | Cell Population | | | |
|---|---|---|---|---|---|
| Code | Type | Disease | Lymphocyte | Monocyte | Granulocyte |

Binding of L32 to Leukemia Patients

| | | | | | | |
|---|---|---|---|---|---|---|
| 1 | 42824 | AML/M2 | 53/1000¹ | 68 | — | — |
| 2 | 42841 | AML/M4 | 330 | 123 | 900² | 280 |
| 3 | 42868 | AML/M2 | 43 | 120 | 1000 | 345 |
| 4 | 42873 | AML/M4 | 24 | 94 | — | 80 |
| 5 | 42874 | AML/M4 | negative | 73 | 35 | negative |
| 6 | 42902 | AML/M6 | 26 | 78 | — | — |
| 7 | 42933 | AML/M5 | 200 | 18 | | |
| 8 | 42939 | AML/M2 | 57 | 110 | — | 160 |
| 9 | 42946 | AML/M0 | negative | 98 | — | — |
| 10 | R0298 | AML/M0 | 23 | 260 | — | — |
| 12 | R9849 | AML/M4 | 470 | 260 | — | 340 |
| 13 | KAM095 | RAEB | 30 | 83 | — | 240 |
| 14 | R5440-7 | AML/M3 | 200 | 140 | — | — |
| 15 | R0376 | AML/M1/M2 | 102 | 65 | 295 | 186 |
| 16 | KAM096 | AML/M2 | 36 | 41 | — | — |
| 17 | KAM108 | Relapse | 94³ | 143 | 360 | 280 |
| | | AML | | | | |
| 18 | KAM109 | AML/M5 | 91 | 18 | — | 38 |
| 19 | KMM097 | MM | 100 | 27 | — | — |
| 20 | 42934 | MM | 650 | 130 | 560 | 150 |
| 21 | 42938 | MM | 550 | 110 | 360 | 160 |
| 22 | KPC105 | Plasmacytoma | 340 | 330 | — | 280 |
| 23 | KBC098 | B-CLL | 37 | 154 | — | 240 |
| 24 | KBC100 | B-CLL | 10 | 46 | — | 68 |
| 25 | KBC101 | B-CLL | negative⁴ | 140 | 280 | 190 |
| 26 | KBC102 | B-CLL | negative | — | — | 110 |
| 27 | KBC103 | B-CLL | 13 | 46 | 300 | 230 |
| 28 | KBC104 | B-CLL | negative⁴ | 60 | 260 | negative |
| 29 | KBC105 | B-CLL | 70 | 250 | — | 260 |
| 30 | KBC106 | B-CLL | negative | 68 | 200 | 140 |
| 31 | KBC107 | B-CLL | 17 | 165 | 420 | 270 |
| 32 | R3093-7 | B-ALL | negative | 41 | 490 | 90 |
| 33 | R313-0 | B-ALL | negative | 26 | — | 100 |

Binding of Y1 scFv to Leukemia Patients

| | | | | | | |
|---|---|---|---|---|---|---|
| 1 | 42824 | AML/M2 | neg./87¹ | 50 | | |
| 2 | 42841 | AML/M4 | 125 | 59 | 270² | 126 |
| 3 | 42868 | AML/M2 | 15 | 53 | 100 | 180 |
| 4 | 42873 | AML/M4 | 12 | 38 | — | 43 |
| 5 | 42874 | AML/M4 | negative | 36 | 30 | negative |
| 6 | 42902 | AML/M6 | 19 | 24 | — | — |
| 7 | 42933 | AML/M5 | 62 | 10 | | |
| 8 | 42939 | AML/M2 | 8 | 18 | — | 51 |
| 9 | 42946 | AML/M0 | negative | 27 | — | — |
| 10 | R0298 | AML/M0 | negative | 43 | | |
| 12 | R9849 | AML/M4 | 230 | 160 | — | 225 |
| 13 | KAM095 | RAEB | 12 | 20 | — | 74 |
| 14 | R5440-7 | AML/M3 | 95 | 43 | | |
| 15 | R0376 | AML/M1/M2 | 29 | 18 | 100 | 63 |
| 16 | KAM096 | AML/M2 | 17 | 15 | — | — |
| 17 | KAM108 | Relapse | 15³ | 20 | 110 | 120 |
| | | AML | | | | |
| 18 | KAM109 | AML/M5 | 40 | 14 | — | 18 |
| 19 | KMM097 | MM | 70 | 13 | — | — |
| 20 | 42934 | MM | 170 | 30 | 160 | 50 |
| 21 | 42938 | MM | 184 | 30 | 128 | 68 |
| 22 | KPC105 | Plasmacytoma | 117 | 116 | — | 100 |
| 23 | KBC098 | B-CLL | 21 | 70 | — | 140 |
| 24 | KBC100 | B-CLL | 19 | 17 | — | 27 |
| 25 | KBC101 | B-CLL | negative⁴ | 29 | 84 | 47 |
| 26 | KBC102 | B-CLL | negative | — | — | 60 |
| 27 | KBC103 | B-CLL | negative | 20 | 86 | 110 |
| 28 | KBC104 | B-CLL | negative⁴ | 10 | 84 | negative |
| 29 | KBC105 | B-CLL | 46 | 117 | — | 133 |
| 30 | KBC106 | B-CLL | negative | 41 | 98 | 65 |
| 31 | KBC107 | B-CLL | 9.5 | 79 | | 126 |
| 32 | R3093-7 | B-ALL | negative | | | |
| 33 | R313-0 | B-ALL | negative | | | |

¹Two disease populations
²Of the disease cells
³Peripheral blood contains 2.5% CD34+ cells
⁴The cells are CD19+/CD5−

Of the seventeen AML samples tested, fifteen (88%) were positive for binding by L32 with ten samples (59%) showing high affinity binding (avr. Geo mean 165) and five samples (29%) showing medium affinity binding (avr. Geo mean 28). Thirteen (76%) of the AML samples were also positive for Y1 binding, but comparative analysis (Table 16) indicates that L32 average binding affinity is always markedly higher than that of Y1 in the same sample population. In general, about 50% of AML patient samples at stage M2 exhibit binding by both L32 and Y1. At stage M3 and greater, about 90% of samples bind both antbodies, with varying degrees of affinity (data not shown).

Of the nine B-CLL samples tested, four (44%) were positive for binding by L32 with one sample (11%) showing high afininty binding (Geo mean 70), one sample (11%) showing medium afininty binding (Geo mean 37) and two samples (22%) showing low affinity binding (avr. Geo mean 15). Three (33%) of the B-CLL samples tested were also positive for Y1 binding, but comparative analysis (Table 16) indicates that L32 average binding affinity is always markedly higher than that of Y1 in the same sample population.

Of the four MM and plastmacytoma samples tested, all showed high afinity binding to both L32 and Y1, but affinity of L32 was greater than that of Y1 (avr. Geo mean 410 vs. avr. Geo mean 135, respectively). Of the two B-ALL samples tested, both were negative for binding of both L32 and Y1.

TABLE 16

| | |
|---|---|
| AML | B-CLL |

| | L32 | Y1 | L32 | Y1 |
|---|---|---|---|---|
| High Geo Mean | | | | |
| Disease | 165 | 61 | 70 | 46 |
| Lymphocytes | 117 | 50 | 107 | 42 |
| Monocytes | 638 | 145 | 323 | 58 |
| Granulocytes | 239 | 110 | 169 | 70 |
| Medium Geo Mean | | | | |
| Disease | 28 | 12 | 37 | 21 |
| Lymphocytes | 41 | 15 | 26 | 9 |
| Monocytes | 35 | 30 | Neg. | Neg. |
| Granulocytes | Neg. | Neg. | Neg. | Neg. |
| Low Geo Mean | | | | |
| Disease | None | None | 13 | 9 |
| Lymphocytes | 18 | 12 | Neg. | Neg. |
| Monocytes | Neg. | Neg. | Neg. | Neg. |
| Granulocytes | Neg. | Neg. | Neg. | Neg. |

*The numbers are average Geo Means based on the results depicted in Table 14. High, medum and low affinity group was determined according to L32 data.

The results in Tables 15 and 16 clearly indicate that in the diseased population as well as in the other mature subpopulations (lymphocytes, monocytes and granulocytes) in primary AML and B-CLL blood samples L32 antibody binding is always markedly higher than that of Y1.

We claim:

1. An antibody or fragment thereof that binds to an epitope of PSGL-1, wherein the antibody or fragment thereof has the binding capabilities of an scFv of SEQ ID NO:1.

2. The antibody or fragment thereof of claim 1, wherein the antibody or fragment thereof comprises one heavy chain complementarity determining region (CDR) selected from the group consisting of SEQ ID NO:2, SEQ ID NO:3, and SEQ ID NO:4.

3. The antibody or fragment thereof of claim 2, wherein two heavy chain CDRs are selected from the group consisting of SEQ ID NO:2, SEQ ID NO:3, and SEQ ID NO:4.

4. The antibody or fragment thereof of claim 3, wherein three heavy chain CDRs are selected from the group consisting of SEQ ID NO:2, SEQ ID NO:3, and SEQ ID NO:4.

5. The antibody or fragment thereof of claim 4, wherein the antibody or fragment thereof comprises SEQ ID NO: 1.

6. An antibody or fragment thereof that binds to an epitope of PSGL-1 comprising one heavy chain complementarity determining region (CDR) selected from the group consisting of SEQ ID NO:2, SEQ ID NO:3, and SEQ ID NO:4.

7. The antibody or fragment thereof of claim 6, wherein two heavy chain CDRs are selected from the group consisting of SEQ ID NO:2, SEQ ID NO:3, and SEQ ID NO:4.

8. The antibody or fragment thereof of claim 7, wherein three heavy chain CDRs are selected from the group consisting of SEQ ID NO:2, SEQ ID NO:3, and SEQ ID NO:4.

9. An antibody or fragment thereof that binds to an epitope of PSGL-1 comprising SEQ ID NO:1.

10. The antibody or fragment thereof of claim 1, wherein the antibody or fragment thereof comprises at least one framework variable region from germline DP32.

11. The antibody or fragment thereof of claim 1, wherein the antibody or fragment thereof is a substantially circular or looped peptide or polypeptide.

12. The antibody or fragment thereof of claim 1, wherein the epitope comprises at least one sulfated moiety.

13. The antibody or fragment thereof of claim 1, wherein the antibody or fragment thereof binds two or more epitopes, each epitope comprising one or more sulfated tyrosine residues

14. The antibody or fragment thereof of claim 13, wherein each epitope comprises at least one cluster of two or more acidic amino acids.

15. The antibody or fragment thereof of claim 1, wherein the antibody or fragment thereof cross-reacts with two or more epitopes, each epitope having one or more sulfated tyrosine residues.

16. The antibody or fragment thereof of claim 15, wherein each epitope comprises at least one cluster of two or more acidic amino acids.

17. The antibody or fragment thereof of claim 1, wherein the antibody or fragment thereof binds to an epitope on at least one cell type selected from the group consisting of T-ALL cells, AML cells, B-leukemia cells, B-CLL, and multiple myeloma cells.

18. The antibody or fragment thereof of claim 1, wherein the antibody or fragment thereof binds to an epitope on a lipid, carbohydrate, peptide, glycolipid, glycoprotein, lipoprotein, and/or lipopolysaccharide molecule.

19. The antibody or fragment thereof of claim 1, wherein the antibody or fragment thereof is coupled to or complexed with an agent selected from the group consisting of anti-cancer, anti-leukemic, anti-metastasis, anti-neoplastic, anti-disease, anti-adhesion, anti-thrombosis, anti-restenosis, anti-autoimmune, anti-aggregation, anti-bacterial, anti-viral, and anti-inflammatory agents.

20. The antibody or fragment thereof of claim 19, wherein the agent is an anti-viral agent selected from the group consisting of acyclovir, ganciclovir and zidovudine.

21. The antibody or fragment thereof of claim 19, wherein the agent is an anti-thrombosis/anti-restenosis agent selected from the group consisting of cilostazol, dalteparin sodium, reviparin sodium, and aspirin.

22. The antibody or fragment thereof of claim 19, wherein the agent is an anti-inflammatory agent selected from the group consisting of zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid.

23. The antibody or fragment thereof of claim 19, wherein the agent is an anti-autoimmune agent selected from the group consisting of leflunomide, denileukin diftitox, subreum, WinRho SDF, defibrotide, and cyclophosphamide.

24. The antibody or fragment thereof of claim 19, wherein the agent is an anti-adhesion/anti-aggregation agent selected from the group consisiting of limaprost, clorcromene, and hyaluronic acid.

25. The antibody or fragment thereof of claim 19, wherein the agent is selected from the group consisting of toxins, radioisotopes, imaging agents, and pharmaceutical agents.

26. The antibody or fragment thereof of claim 25, wherein the toxin is selected from the group consisting of gelonin, Pseudomonas exotoxin (PE), PE40, PE38, ricin, and modifications and derivatives thereof.

27. The antibody or fragment thereof of claim 25, wherein the radioisotope is selected from the group consisting of gamma-emitters, positron-emitters, x-ray emitters, beta-emitters, and alpha-emitters.

28. The antibody or fragment thereof of claim 25, wherein the radioisotope is selected from the group consisting of ¹¹¹indium, ¹¹³indium, ^{99m}rhenium, ¹⁰⁵rhenium, ¹⁰¹rhenium, ^{99m}technetium, ^{121m}tellurium, ^{122m}tellurium, ^{125m}telluriunm ¹⁶⁵thulium, ¹⁶⁷thulium ¹⁶⁸thulium ¹²³iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵ruthenium ¹⁰⁷mercury, ²⁰³mercury, ⁶⁷gallium and ⁶⁸gallium.

29. The antibody or fragment thereof of claim 25, wherein the pharmaceutical agent is an anthracyline.

30. The antibody or fragment thereof of claim 29, wherein the anthracyline is selected from the group consisting of doxorubicin, daunorubicin, idarubicin, detorubicin, carminomycin, epirubicin, esorubicin, morpholinodoxorubicin, morpholinodaunorubicin, and methoxymorpholinyldoxorubicin.

31. The antibody or fragment thereof of claim 25, wherein the pharmaceutical agent is selected from the group consisting of cis-platinum, taxol, calicheamicin, vincristine, cytarabine (Ara-C), cyclophosphamide, prednisone, fludarabine, chlorambucil, interferon alpha, hydroxyurea, temozolomide, thalidomide and bleomycin, and derivatives and combinations thereof.

32. The antibody or fragment thereof of claim 19, wherein the antibody or fragment thereof is coupled to or complexed with a vehicle or carrier that can be coupled or complexed to more than one agent.

33. The antibody or fragment thereof of claim 32, wherein the vehicle or carrier is selected from the group consisting of dextran, lipophilic polymers, HPMA, and liposomes, and derivatives and modifications thereof.

34. An isolated epitope comprising an amino acid sequence that binds to the antibody or fragment thereof of claim 1.

35. The isolated epitope of claim 34, wherein the isolated epitope comprises at least one sulfated moiety.

36. The isolated epitope of claim 35, wherein the sulfated moiety is a sulfated tyrosine.

37. The isolated epitope of claim 34, wherein the isolated epitope comprises a cluster of negatively charged amino acids.

38. The isolated epitope of claim 37, wherein the cluster comprises amino acids 1 and 17 of mature PSGL-1.

39. An isolated or purified polynucleotide encoding the antibody or fragment thereof of claim 1.

40. An expression vector comprising the polynucleotide sequences of claim 39.

41. A recombinant host cell comprising the expression vector of claim 40.

42. The recombinant host cell of claim 41, or a progeny thereof wherein the cell expresses the antibody or fragment thereof.

43. A method of producing a recombinant cell comprising transfecting a cell with the expression vector of claim 40.

44. A method of producing an antibody or fragment thereof comprising culturing the cell of claim 41 under conditions permitting expression of the antibody or fragment thereof.

45. The method of claim 44, wherein the method further comprises isolating or purifying the antibody or fragment thereof from the cell or medium of the cell.

46. A pharmaceutical composition comprising an antibody or fragment thereof of claim 1 and a pharmaceutically acceptable carrier.

47. A diagnostic, prognostic, or staging kit comprising an antibody or fragment thereof of claim 1 and an imaging agent.

48. The diagnostic, prognostic, or staging kit of claim 47, wherein the imaging agent is a radioactive isotope.

49. A method of treating a disease comprising administering to a patient in need thereof a pharmaceutical composition of claim 46.

50. A method of treating cell rolling comprising administering to a patient in need thereof a pharmaceutical composition of claim 46.

51. A method of ameliorating the effects of inflammation, preventing inflammation, treating inflammation, or inhibiting the progress of inflammation comprising administering to a patient in need thereof a pharmaceutical composition of claim 46.

52. A method of treating an infection comprising administering to a patient in need thereof a pharmaceutical composition of claim 46.

53. The method of claim 52, wherein the infection is caused by HIV.

54. The method of claim 52, wherein the administration prevents cell entry of HIV.

55. A method of treating an auto-immune disease comprising administering to a patient in need thereof a pharmaceutical composition of claim 46.

56. A method of treating metastasis comprising administering to a patient in need thereof a pharmaceutical composition of claim 46.

57. A method of treating growth and/or replication of tumor cells comprising administering to a patient in need thereof a pharmaceutical composition of claim 46.

58. A method of increasing the mortality rate of tumor cells comprising administering to a patient in need thereof a pharmaceutical composition of claim 46.

59. A method of treating growth and/or replication of leukemia cells comprising administering to a patient in need thereof a pharmaceutical composition of claim 46.

60. A method of increasing the mortality rate of leukemia cells comprising administering to a patient in need thereof a pharmaceutical composition of claim 46.

61. A method of altering the susceptibility of diseased cells to damage by anti-disease agents comprising administering to a patient in need thereof a pharmaceutical composition of claim 46.

62. A method of increasing the susceptibility of tumor cells to damage by anti-cancer agents comprising administering to a patient in need thereof a pharmaceutical composition of claim 46.

63. A method of increasing the susceptibility of leukemia cells to damage by anti-cancer agents comprising administering to a patient in need thereof a pharmaceutical composition of claim 46.

64. A method of inhibiting increase in number of tumor cells in a patient having a tumor comprising administering to a patient in need thereof a pharmaceutical composition of claim 46.

65. A method of decreasing number of tumor cells in a patient having tumor comprising administering to a patient in need thereof a pharmaceutical composition of claim 46.

66. A method of inhibiting increase in number of leukemia cells in a patient having leukemia comprising administering to a patient in need thereof a pharmaceutical composition of claim 46.

67. A method of decreasing number of leukemia cells in a patient having leukemia comprising administering to a patient in need thereof a pharmaceutical composition of claim 46.

68. A method of eliciting antibody dependent cell-mediated cytotoxicity (ADCC) comprising administering to a patient in need thereof a pharmaceutical composition of claim 46.

69. A method of stimulating a natural killer (NK) cell or a T cell comprising administering to a patient in need thereof a pharmaceutical composition of claim 46.

70. A method of diagnosing or prognosing a disease in a patient comprising
providing a sample containing a cell from the patient and
determining whether the antibody or fragment thereof of claim 1 binds to the cell of the patient,
thereby indicating that the patient is at risk for or has the disease.

71. A method of diagnosing or prognosing inflammation in a patient comprising
providing a sample containing a cell from the patient and
determining whether the antibody or fragment thereof of claim 1 binds to the cell of the patient,
thereby indicating that the patient is at risk for or has inflammation.

72. A method of diagnosing or prognosing an infection in a patient comprising
providing a sample containing a cell from the patient and
determining whether the antibody or fragment thereof of claim 1 binds to the cell of the patient,
thereby indicating that the patient is at risk for or has an infection.

73. The method of claim 72, wherein the infection is caused by HIV.

74. A method of diagnosing or prognosing an auto-immune disease in a patient comprising
providing a sample containing a cell from the patient and
determining whether the antibody or fragment thereof of claim 1 binds to the cell of the patient,
thereby indicating that the patient is at risk for or has an auto-immune disease.

75. A method of diagnosing, prognosing, or staging metastasis in a patient comprising
providing a sample containing a cell from the patient and
determining whether the antibody or fragment thereof of claim 1 binds to the cell of the patient,
thereby indicating that the patient is at risk for or has metastasis.

76. A method of diagnosing, prognosing, or staging a tumor cell in a patient comprising
providing a sample containing a cell from the patient and
determining whether the antibody or fragment thereof of claim 1 binds to the cell of the patient,
thereby indicating that the patient is at risk for or has a tumor cell.

77. A method of diagnosing, prognosing, or staging leukemia in a patient comprising
providing a sample containing a cell from the patient and
determining whether the antibody or fragment thereof of claim 1 binds to the cell of the patient,
thereby indicating that the patient is at risk for or has leukemia.

78. A method of purging tumor cells from a patient comprising
providing a sample containing cells from the patient and
incubating the cells from the patient with an antibody or fragment thereof of claim 1.

79. The method of claim 78, wherein the purging occurs ex vivo.

80. Use of a pharmaceutical composition of claim 46 in the manufacture of a medicament for treating a disease.

81. The use of claim 80, wherein the disease is selected from the group consisting of cell rolling, inflammation, an auto-immune disease, an infection, metastasis, growth and/or replication of tumor cells, and growth and/or replication of leukemia cells.

82. A pharmaceutical composition of claim 46 for use in the manufacture of a medicament for treating a disease.

83. The pharmaceutical composition of claim 82, wherein the disease is selected from the group consisting of cell rolling, inflammation, an auto-immune disease, an infection, metastasis, growth and/or replication of tumor cells, and growth and/or replication of leukemia cells.

84. A process for producing an antibody or fragment thereof comprising the steps of:
providing a phage display library;
providing at least two molecules or cells that binds to an antibody or fragment thereof having the binding capabilities of an scFv antibody or fragment thereof fragment of SEQ ID NO: 1;
panning the phage display library for a phage particle displaying an oligopeptide or polypeptide that binds to at least two of the molecules or cells; and
producing an antibody or fragment thereof comprising an antibody or fragment thereof or binding fragment thereof comprising the peptide or polypeptide that binds to at least two of the molecules or cells.

85. An antibody or fragment thereof produced according to the process of claim 84.


SEQUENCE LISTING (8 sequences)

SEQ ID NO: 1 - protein, 280 aa, Homo sapiens
MKYLLPTAAAGLLLLAAQPAMAEVQLVESGGGVVRPGGSLRLSCAASGFTFDLNPKVKHMWVRQAPGKGLEWVSGINWNGGSTGYADSVKGRFTISRDNAKNSLYLQMNSLRAEDTAVYYCARMRAPVIWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAEQKLISEEDLNGAA

SEQ ID NO: 2 - protein, 6 aa, Homo sapiens
MRAPVI

SEQ ID NO: 3 - protein, 16 aa, Homo sapiens
GINWNGGSTGYADSVK

SEQ ID NO: 4 - protein, 8 aa, Homo sapiens
LNPKVKHM

SEQ ID NO: 5 - protein, 7 aa, Homo sapiens
LRGGNAM

SEQ ID NO: 6 - protein, 11 aa, Homo sapiens
FLTYNSYEVPT

SEQ ID NO: 7 - protein, 9 aa, Homo sapiens
TNWYLRPLN

SEQ ID NO: 8 - protein, 10 aa, Homo sapiens
EQKLISEEDL